(19) United States
(12) Patent Application Publication
(10) Pub. No.: US 2022/0313566 A1
(43) Pub. Date: Oct. 6, 2022

(54) MULTILAYER MATERIAL FOR SCREENING OUT ULTRAVIOLET, COMPOSITION COMPRISING SAME, PROCESS FOR TREATING KERATIN MATERIALS USING SAME, AND PROCESS FOR PREPARING THE MATERIAL

(71) Applicant: L'OREAL, Paris (FR)

(72) Inventors: Andres CARDOZO PEREZ, Saint-Ouen (FR); Antoine MONTAUX-LAMBERT, Aulnay-Sous-Bois (FR)

(21) Appl. No.: 17/607,926

(22) PCT Filed: May 6, 2020
(86) PCT No.: PCT/EP2020/062627

§ 371 Date: Nov. 1, 2021

(30) Foreign Application Priority Data
May 9, 2019 (FR) FR1904826

(51) Int. Cl. A61K 8/02; A61K 8/29; A61K 8/25; A61Q 17/04; A61K 8/19

(52) U.S. Cl. CPC A61K 8/0245; A61K 8/29; A61K 8/25; A61Q 17/04; A61K 8/19

(57) ABSTRACT

The invention relates to i) a multilayer material; ii) a process for preparing said multilayer materials; iii) a cosmetic composition comprising one or more multilayer materials; iv) a process for treating keratin materials, notably human keratin materials such as the skin; v) the use of multilayer material for screening out ultraviolet (UV) rays. Said multilayer material has an odd number N of layers: ▪comprising at least three layers, each layer of which consists of a material A or of a material B different from A, said successive layers A and B being alternated and two adjacent layers having different refractive indices; ▪for which the thickness of each layer obeys the mathematical formula (I) below: [x/y/(αx/y)ₐ/x] in which formula (I): x is the thickness of the inner and outer layer; y is the thickness of the layer adjacent to the inner layer αx or the outer layer x; α is an integer or fraction and α=2±0 to 15%, preferably α=2±0 to 10%, more preferentially α=2±0 to 5%, the intermediate odd layers (αx) have a double thickness±0 to 15% of the thickness of said outer layers x; and a represents an integer greater than or equal to 0, connected to the number of alternated layers N such that a=(N−3)/2; it being understood that: ▪preferably, x has a different thickness from y; ▪when several layers are of thickness x, this means that each layer has a thickness x±0 to 15%, preferably±0 to 10%, more preferentially±0 to 5%; ▪when several layers are of thickness y, this means that each layer has a thickness y±0 to 15%, preferably±0 to 10%, more preferentially±0 to 5%; and ▪when several layers are of thickness α x, this means that each layer has a thickness α x±0 to 15%, preferably±0 to 10%, more preferentially±0 to 5%.

The subjects of the invention are i) a multilayer material of particular structure with an odd number of layers, comprising at least three layers, said successive layers of which are alternated and in which the adjacent layers have different refractive indices, ii) a process for preparing said multilayer material; iii) a composition, notably a cosmetic composition, comprising one or more multilayer materials; iv) a process for treating keratin materials, notably human keratin materials such as the skin, using at least said multilayer material i) or said composition iii); v) the use of the multilayer material for screening out ultraviolet (UV) rays.

Various types of UV-screening agents are known in the prior art, for example inorganic UV-screening agents also known as mineral screening agents, such as titanium dioxide (TiO2) and zinc oxide (ZnO), and organic UV-screening agents such as benzophenone derivatives and cinnamic derivatives.

On the daily sun-protection and photoprotection market, photoprotection using mineral UV-screening agents is a very important expectation of consumers throughout the world. Many consumers consider mineral sunscreens to be safer for sensitive skins. TiO₂ and ZnO are the most common mineral sun-protection agents in mineral photoprotection products. However, the efficiency of TiO₂ and ZnO is limited, in particular in the UV-A wavelength range (320 nm to 400 nm). In addition, to achieve high sun protection factor (SPF) values (for example 50), large amounts of UV-screening agents are necessary, which induces substantial whitening effects and/or unpleasant sensations after application to the skin.

(In)organic materials are thus sought which are capable of efficiently blocking UV rays (i.e. materials with a low UV ray transmission), in particular in the UVA range, and which have high transparency to visible light (i.e. materials with a high transmission of rays between 400 and 780 nm), and which do not whiten after application.

Among the UV-screening agents used in cosmetics, it is known practice to use multilayer particles. For example, Japanese patent JP 3986304 describes a multilayer pigment for protecting against ultraviolet rays. WO 2014/150846 A1 mentions cosmetic applications for pigments which reflect UV rays on a substrate. WO 2003/063616 A1 describes the use of multilayer pigments based on substrates and based on minerals in plate form, for coloring pharmaceutical and food products. US 2005/0176850 A1 mentions interference pigments based on a coating of TiO₂ on transparent substrate flakes, said substrate having a thickness of between 20 nm and 2 μm.

In addition, JP-A-2003/171575 describes an interference pigment with stratified interference for protecting against UV rays, which comprises a lamellar or flatter pigment covered with alternating layers including at least three layers of a metal oxide with a high refractive index and of a metal oxide with a low refractive index. JP-A-2014-811 describes a process for manufacturing a substrate-free multilayer thin film.

US 2006/0027140 describes a multilayer interference pigment comprising a platelet-shaped or lamellar substrate which consists of successive alternating layers of materials with high and low refractive indices, said interference pigment having a total thickness of ≤1 μm.

However, these screening agents are not always satisfactory in terms of screening out UV rays. They notably do not have a very narrow filtration front and a high transmittance region in the visible wavelengths making them highly transparent, i.e. they do not have a “steep” filtration front between the low transmittance region (UV) and the high transmittance region.

Novel materials are also sought which comprise few layers to reduce the manufacturing costs, while at the same time improving the sun-protection properties notably in the UVA and UVB ranges.

In addition, there is a need to provide a material which screens out UV rays, which is designed to be able to screen out only a fraction of the light radiation, i.e. target light, such as the wavelength range of UV and light radiation, such as UVA and UVB.

One of the objects of the present invention is to provide a material for screening out UV rays, which is capable of screening out only UV rays, intrinsically and/or optionally after its implementation.

To do this, the material intrinsically has a very narrow filtration front and/or a very narrow filtration front after its implementation, and a high transmittance range notably for visible wavelengths, above the “cut-off”.

Thus, one of the objects of the invention is to provide a material for screening out UV rays, which is capable of screening out only UV rays, intrinsically and/or optionally after its implementation.

It has been discovered that the material of the invention notably has, as noteworthy optical property, a narrow filtration front between UV and the visible range and a high transmittance in the visible range, i.e. having a transmittance-to-wavelength slope which is “steep”, i.e. greater than 2.5×10⁻³ nm⁻¹, preferably greater than 3×10⁻³ nm⁻¹, more preferentially greater than 4×10⁻³ nm⁻¹.

A subject of the invention is also the use of at least one multilayer material for screening out UV rays, for protecting keratin materials and in particular the skin against UV rays, in particular in the UVA range.

The invention also relates to a composition, in particular a cosmetic composition for antisun care, skin care, hair care and makeup.

The invention also relates to the multilayer material itself.

The invention also relates to a particular method for preparing the multilayer material. The invention also relates to a process for applying said multilayer material to keratin materials such as the skin.

The multilayer material of the invention affords UV protection with high UV-screening properties, exceptional transparency in the visible range (400 to 780 nm) and a cut-off that is well-defined intrinsically and/or during its use, in various modes of application.

The use of such multilayer materials of the invention makes it possible to better screen out UVA (320 nm to 400 nm), in particular for long UVA (340 nm to 400 nm), while at the same time maintaining good transparency in the visible range (400 nm to 780 nm). Furthermore, the use of said multilayer material may also allow good screening of UV-B rays (from 280 to 320 nm).

For the purposes of the present invention and unless otherwise indicated:
the term “filtration front” corresponds to the transition wavelength range between the lowest value and the highest value of the transmittance (cut-off transition range); the term “cut-off wavelength” (λ_{c}, cut-off) means the wavelength value at the center of the filtration front;
the term “transmittance-to-wavelength slope” is defined as follows:

$$[ Math . 1 ]$$

$$Slope = \frac{{Transmittance}_{max} - {Transmittance}_{min}}{{λ}_{max} - {λ}_{min}} = \frac{T r a n s m i t t a n c {e}_{max} - {Transmittance}_{min}}{Filtration front}$$

$$Tran s m i t t a n c {e}_{max} = transmittance value to {λ}_{max}$$

$$Tr {ansmittance}_{min} = transmittance value to {λ}_{min}$$

$${λ}_{c} = cut - off = ( {λ}_{{max}^{-}} {λ}_{min} ) / 2$$

the term “at least one” is equivalent to “one or more”; and
the term “inclusive” for a range of concentrations means that the limits of the range are included in the defined interval.
the term “alkyl” means a linear or branched, saturated hydrocarbon-based radical, comprising between 1 and 20 carbon atoms, preferably between 1 and 6 carbon atoms;
the term “alkylene” means a linear or branched, saturated divalent hydrocarbon-based radical, comprising from 1 to 20 carbon atoms, preferably between 1 and 6 carbon atoms;
the term “alkenyl” means a linear or branched, unsaturated hydrocarbon-based radical, comprising between 2 and 20 carbon atoms, preferably between 2 and 6 carbon atoms, and from 1 to 3 conjugated or non-conjugated unsaturations;
the term “aryl” means a cyclic unsaturated and aromatic carbon-based radical, comprising one or more rings, at least one of the rings of which is aromatic, and comprising from 5 to 10 carbon atoms, such as phenyl;
   the term “arylene” means an aryl group as defined previously, which is divalent;
the term “(in)organic” means organic or inorganic and preferably inorganic;
the terms “inorganic” and “mineral” are used without distinction.

### Multilayer Material

The first subject of the invention is a multilayer material having an odd number N of layers:
comprising at least three layers (N greater than or equal to 3), each layer of which consists of a material A or of a material B different from A, said successive layers A and B being alternated and two adjacent layers having different refractive indices;
for which the thickness of each layer obeys the mathematical formula (I) below: [x/y/(αx/y)ₐ/x]
in which formula (I):
x is the thickness of the inner and outer layer;
y is the thickness of the layer adjacent to the inner layer αx or the outer layer x;
α is an integer or fraction and α=2±0 to 15%, preferably α=2±0 to 10%, more preferentially α=2±0 to 5%,
the intermediate odd layers (αx) have a double thickness±0 to 15% of the thickness of said outer layers x; and
a represents an integer greater than or equal to 0, connected to the number of alternated layers N such that a=(N−3)/2;
it being understood that:
preferably, x is a different thickness from y;
when several layers are of thickness x, this means that each layer has a thickness x±0 to 15%, preferably±0 to 10%, more preferentially±0 to 5%;
when several layers are of thickness y, this means that each layer has a thickness y±0 to 15%, preferably±0 to 10%, more preferentially±0 to 5%; and
when several layers are of thickness α x, this means that each layer has a thickness α x±0 to 15%, preferably±0 to 10%, more preferentially±0 to 5%.

### Chemical Composition of the Superposed Alternated Layers

### Forms of the Compounds Constituting the Superposed Layers of the Material:

The multilayer material is a superposition of layers that are different from each other, each layer consisting of a material A or of a material B different from A, said successive layers being alternated and two adjacent layers having different refractive indices. Thus, if the multilayer compound includes three layers, A may constitute the outer layer and the multilayer material is represented by the stack A/B/A or else B may constitute the outer layer and the multilayer material is represented by the stack B/A/B. Similarly, if the multilayer compound includes five layers, A may constitute the outer layer and the multilayer material is represented by the stack A/B/A/B/A or else B may constitute the outer layer and the multilayer material is represented by the stack B/A/B/A/B.

Compounds A and B are (in)organic materials with different refractive indices. Preferably, the difference in refractive index between material A and material B is at least 0.3; in particular, this difference is between 0.3 and 2, preferably between 0.4 and 2, more preferentially between 0.5 and 1.8, even more preferentially between 0.6 and 1.5 or even more preferably between 0.7 and 1.3.

According to a preferred form of the invention, the materials A and B are inorganic materials.

According to one embodiment, the outer layer is a layer with a lower refractive index than the adjacent layer.

According to another embodiment, the outer layer has a higher refractive index than the adjacent layer.

The thickness of each layer is particularly between 5 and 500 nm, and more preferentially between 10 and 200 nm.

The stack of the various layers is such that the thickness of each layer obeys the mathematical formula (I) defined previously.

The (in)organic material A (or, respectively, B) may consist of a single pure compound or of a mixture of inorganic compounds, or else of a mixture of organic and inorganic compounds, or else a mixture of organic compounds, it being understood that A and B have different refractive indices as described previously.

According to a particular form of the invention, A and B are different and A and B consist, independently, of a pure inorganic compound or of a mixture of inorganic compounds, it being understood that A and B have different refractive indices as described previously.

According to a preferred variant of the invention, A and B are different and A and B consist of a pure inorganic compound, it being understood that A and B have different refractive indices as described previously.

When the materials A and B consist of inorganic materials in pure form or as a mixture, these inorganic compounds constituting A and B are in particular chosen from: germanium (Ge), gallium antimonide (GaSb), tellurium (Te), indium arsenide (InAs), silicon (Si), gallium arsenide (GaAs), indium phosphide (InP), gallium phosphide (GaP), graphite (C), chromium (Cr), zinc telluride (ZnTe), zinc sulfate (ZnSO₄), vanadium (V), arsenic selenide (As₂Se₃), rutile titanium dioxide (TiO₂), copper aluminum diselenide (CuAlSe₂), perovskite calcium titanate (CaTiO₃), tin sulfide (SnS), zinc selenide (ZnSe), anatase titanium dioxide (TiO₂), cerium oxide (CeO₂), gallium nitride (GaN), tungsten (W), manganese (Mn), titanium dioxide notably vacuum-deposited (TiO₂), diamond (C), niobium oxide (Nb₂O₃), niobium pentoxide (Nb₂O₅), zirconium oxide (ZrO₂), sol-gel titanium dioxide (TiO₂), zinc sulfide (ZnS), silicon nitride (SiN), zinc oxide (ZnO), aluminum (Al), hafnium oxide (HfO₂), corundum aluminum oxide or corundum (Al₂O), aluminum oxide (Al₂O₃), yttrium oxide (Y₂O₃), periclase magnesium oxide (MgO), polysulfone, sodium aluminum fluoride (Na₃AlF), lead fluoride (PbF₂), mica, aluminum arsenide (AlAs), sodium chloride (NaCl), sodium fluoride (NaF), silica (SiO₂), barium fluoride (BaF₂), potassium fluoride (KF), vacuum-deposited silica (SiO₂), indium tin oxide (ITO), strontium fluoride (SrF₂), calcium fluoride (CaF₂), lithium fluoride (LiF), magnesium fluoride (MgF₂), bismuth oxychloride (BiOCl), bismuth ferrite (BiFeO₃), boron nitride (NB), and (bi)carbonate such as calcium carbonate (CaCO₃).

According one interesting embodiment of the invention compounds constituting A and B are more particularly chosen from TiO₂+SiO₂, or TiO₂+MgF₂, or TiO₂+BaF₂, TiO₂+MgO, TiO₂+CaCO₃, Nb₂O₅+SiO₂, or Nb₂O₅+MgF₂, or Nb₂O₅+BaF₂, Nb₂O₅+MgO, Nb₂O₅+CaCO₃, ZnO+MgF₂, ZnS+MgF₂).

When A or B contain organic compounds, said compounds are chosen from polystyrene (PS), polycarbonate, urea formaldehyde, styrene-acrylonitrile copolymers, polyether sulfone (PES), polyvinyl chloride (PVC), polyamide nylons notably of 6/6 type, styrene-butadiene copolymers, type II polyamide nylons, multiacrylic polymers such as polymethyl methacrylate, ionomers, polyethylene, polybutylene, polypropylene, cellulose nitrate, acetal homopolymers such as polyformaldehyde, methylpentene polymers, ethylcellulose, cellulose acetatebutyrate, cellulose propionate, cellulose acetate, chlorotrifluoroethylene (CTFE), polytetrafluoroethylene (PTFE), fluorocarbon or polyvinylidene fluoride (F EP), preferably polystyrene.

According to a preferred form of the invention, A and B consist of pure inorganic materials; these inorganic compounds constituting A and B are in particular chosen from: anatase titanium dioxide (TiO₂), titanium dioxide notably vacuum-deposited (TiO₂), sol-gel titanium dioxide (TiO₂), silica (SiO₂), vacuum-deposited silica (SiO₂).

According another embodiment of the invention multilayer material of the invention is a mixture of inorganic material A and organic material B, or a mixture of organic material A and inorganic material B, such as a mixture of A SiO₂ and B PS or A PS and B SiO₂. Especially SiO₂ (in a weight amount range between 60 and 99%, preferably between 80% and 95% such as 90%) polystyrene (PS) (in a weight amount range between 1 and 40%, preferably between 5% and 20% such as 10%).

### Refractive Index of the Compounds Constituting the Superposed Layers of the Material

The multilayer material of the invention has an odd number (N) of layers and comprises at least three layers, the successive layers of which are alternated and in which the layers consist of (in)organic compounds with different refractive indices which preferably differ by at least 0.3.

The chemical compositions of the superposed layers may be represented in the following manner: x/y/αx/y/x or x/y/αx/y/x or x/y/αx/y/αx/y/x or x/y/αx/y/αx/y/x or x/y/αx/y/αx/y/x or x/y/αx/y/αx/y/αx/y/x or x/y/αx/y/αx/y/αx/y/x or x/y/αx/y/αx/y/αx/y/x or x/y/αx/y/αx/y/αx/y/x . . . with x, y, layers with different refractive indices each consisting of pure (in)organic compounds or a mixture of (in)organic compounds and more particularly pure inorganic compounds. All the layers x have the same refractive index as each other, and all the layers y have the same refractive index as each other, and αx as defined previously.

According to a particular embodiment, the adjacent layers are such that one layer consists of (in)organic compound(s) with a refractive index, and the other adjacent layer consists of (in)organic compound(s) with a lower refractive index, i.e. the refractive index value of the layer is higher than the refractive index of the other adjacent layer by at least 0.3.

In particular, the difference in refractive index between the adjacent layers is inclusively between 0.3 and 2, preferably between 0.4 and 2, more preferentially between 0.5 and 1.8, even more preferentially between 0.6 and 1.5 or even more preferentially between 0.7 and 1.3.

### List Of (In)Organic Compounds and Examples of Refractive Indices Constituting the Superposed Layers of the Material:

According to a particular embodiment of the invention, the compounds with a high refractive index (i.e. with a refractive index of greater than or equal to 1.7) are in particular inorganic compounds and preferably chosen from: germanium (formula: Ge; refractive index: 4.0-5.0), gallium antimonide (GaSb; 4.5-5.0), tellurium (Te; 4.6), indium arsenide (InAs; 4.0), silicon (Si; 3.97), gallium arsenide (GaAs; 3.53), indium phosphide (InP; 3.5), gallium phosphide (GaP; 3.31), graphite (C; 3.13), chromium (Cr; 3.0), zinc telluride, zinc sulfate (ZnSO₄; 3.0), (ZnTe; 3.0), vanadium (V; 3), zinc sulfate (ZnSO₄; 2.5-3.0), arsenic selenide (As₂Se₃; 2.8), rutile titanium dioxide (TiO₂; 2.77), CuAlSe₂ (2.75), perovskite calcium titanate (CaTiO₃; 2.74), tin sulfide (SnS; 2.6), zinc selenide (ZnSe; 2.6), anatase titanium dioxide (TiO₂; 2.55), cerium oxide (CeO₂; 2.53), gallium nitride (GaN; 2.5), tungsten (W; 2.5), manganese (Mn; 2.5), titanium dioxide notably vacuum-deposited (TiO₂; 2.5), diamond (2.42), niobium oxide (Nb₂O₃; 2.4), niobium pentoxide (Nb₂O₅; 2.4), zirconium oxide (ZrO₂; 2.36), sol-gel titanium dioxide (TiO₂; 2.36), zinc sulfide (ZnS; 2.3), silicon nitride (SiN; 2.1), zinc oxide (ZnO; 2.01), aluminum (Al; 2.0), hafnium oxide (HfO₂; 1.9-2.0), corundum aluminum oxide or corundum (Al₂O₃; 1.76), aluminum oxide (Al₂O₃; 1.75), yttrium oxide (Y₂O₃; 1.75), periclase magnesium oxide (MgO; 1.74), bismuth oxychloride (BiOCl), bismuth ferrite (BiFeO₃), and boron nitride (NB);.

Two or more compounds with a high refractive index may be used as a mixture, preferably between two and five compounds, particularly two.

Preferably, the compounds with a high refractive index are used pure.

According to a particular embodiment of the invention, the inorganic compounds with a low refractive index, i.e. a refractive index of less than 1.7, are chosen from: polysulfone (1.63), sodium aluminum fluoride (Na₃AlF₆; 1.6), lead fluoride (PbF₂; 1.6), mica (1.56), aluminum arsenide (AlAs; 1.56), sodium chloride (NaCl; 1.54), sodium fluoride (NaF; 1.5), silica (SiO₂; 1.5), barium fluoride (BaF₂; 1.5), potassium fluoride (KF; 1.5), vacuum-deposited silica (SiO₂; 1.46), indium tin oxide (ITO; 1.46), lithium fluoride (LiF₄; 1.45), strontium fluoride (SrF₂; 1.43), calcium fluoride (CaF₂; 1.43), lithium fluoride (LiF; 1.39), magnesium fluoride (MgF₂; 1.38), and the organic compounds are chosen from polyetherimide (PEI; 1.6), polystyrene (PS; 1.6), PKFE (1.6), polycarbonate (1.58), urea formaldehyde (1.54-1.58), styrene-acrylonitrile copolymer (1.56), polyether sulfone (PES; 1.55), polyvinyl chloride (PVC,1.55), type 6/6 polyamide nylons (1.53), styrene butadiene (1.52), type II polyamide nylons (1.52), multiacrylic polymers (1.52), ionomers (1.51), polyethylene (1.5), polymethyl methacrylate (PMMA. 1.5), polybutylene (1.50), cellulose acetate (1.46-1.50), polyallomer (PA; 1.49), polypropylene (1.49), cellulose nitrate (1.49), acetal homopolymer (1.48), methylpentene polymer (1.48), ethylcellulose (1.47), cellulose acetate butyrate (1.46), cellulose propionate (1.46), cellulose acetate (1.46), chlorotrifluoroethylene (CTFE; 1.42), polytetrafluoroethylene (PTFE; 1.35), fluorocarbon or polyvinylidene fluoride (FEP; 1,34) and (bi)carbonate such as calcium carbonate (CaCO₃);.

Two or more compounds with a low refractive index may be used as a mixture, preferably between two and five compounds, more preferentially two.

The material according to the invention preferably contains layers y consisting of compounds with a lower refractive index than x; preferentially chosen from metal oxides, halides and carbonates, more particularly metal oxides of metals, and carbonates which are in the Periodic Table of the Elements in columns IIA, IIIB, IVB and VIIB; more particularly, the metal oxides or carbonates with a low refractive index are chosen from CaCO₃, SiO₂, MgO and ITO, and fluorides, notably Na₃AIF₆, MgF₂, PbF₂, CaF₂, KF, LiF, BaF₂, NaF and SrF₂, and preferentially chosen from BaF₂, MgF₂, CaCO₃, ITO, SiO₂ and MgO, more preferentially CaCO₃, SiO₂ or MgO, even more preferentially MgF₂, CaCO₃, SiO₂.

According to a preferred embodiment of the invention, the compounds with a high refractive index are chosen from in which the layers y consist of compounds with a higher refractive index than x, in particular inorganic compounds and are preferably chosen from metal oxides, particularly metal oxides of metals which are in the Periodic Table of the Elements in columns IIIA, IVA, VA, IIIB and lanthanides, more particularly chosen from the following metal oxides: TiO₂, CeO₂, Nb₂O₃, Nb₂O₅, HfO₂, Al₂O₃, Y₂O₃ and ZrO₂, more preferentially Nb₂O₅, TiO₂, CeO₂ and even more preferentially TiO₂, Nb₂O₅.

Preferably, the compounds with a low refractive index are used pure. According to a preferred embodiment of the invention, the compounds with a high refractive index are chosen from metal oxides, particularly the metal oxides of metals which are in the Periodic Table of the Elements in columns IIIA, IVA, VA and IIIB and the lanthanides, more particularly chosen from the following metal oxides: TiO₂, CeO₂, Nb₂O₃, Nb₂O₅, HfO₂, Al₂O₃, Y₂O₃ and ZrO₂, more particularly TiO₂, Nb₂O₅, CeO₂ and preferentially TiO₂, Nb₂O₅, more preferentially TiO₂, CeO₂ and even more preferentially TiO₂.

According to an advantageous embodiment of the invention, the compounds with a low refractive index are chosen from metal oxides and halides, more particularly metal oxides of metals which are in the Periodic Table of the Elements in columns IIA, IVB and VIIB; more particularly, the metal oxides with a low refractive index are chosen from SiO₂, MgO and ITO, and fluorides, notably Na₃AlF₆, MgF₂, PbF₂, CaF₂, KF, LiF, BaF₂, NaF and SrF₂, and preferentially chosen from ITO, SiO₂ and MgO, more preferentially SiO₂ or MgO, even more preferentially SiO₂.

According to yet another particular embodiment of the invention, the adjacent layers have a high refractive index and the difference in refractive index between the adjacent layers is inclusively between 0.3 and 2, preferably between 0.4 and 2, more preferentially between 0.5 and 1.8, even more preferentially between 0.6 and 1.5 or even more preferentially between 0.7 and 1.3.

According to yet another embodiment of the invention, the adjacent layers have a low refractive index and the difference in refractive index between the adjacent layers is inclusively between 0.3 and 2, preferably between 0.4 and 2, more preferentially between 0.5 and 1.8, even more preferentially between 0.6 and 1.5 or even more preferentially between 0.7 and 1.3.

### Number N of Superposed Layers of the Material:

The multilayer material of the invention comprises at least three layers (N greater than or equal to 3). According to a particular mode of the invention, the number of layers N is odd and between 3 and 17, more particularly between 3 and 13 and even more particularly between 3 and 9.

### Thickness of the Layers of the Material:

Relationship between the Layers of the Material of the Invention and the Thickness of the Layers

The multilayer material of the invention is a material with an odd number N of layers:
comprising at least three layers (N greater than or equal to 3), each layer of which consists of a material A or of a material B different from A, said successive layers A and B being alternated and two adjacent layers having different refractive indices;
for which the thickness of each layer obeys the mathematical formula (I) below: [x/y/(αx/y)ₐ/x]
in which formula (I):
x is the thickness of the inner and outer layer;
y is the thickness of the layer adjacent to the inner layer αx or the outer layer x;
α is an integer or fraction and α=2±0 to 15%, preferably α=2±0 to 10%, more preferentially α=2±0 to 5%,
the intermediate odd layers (αx) have a double thickness±0 to 15% of the thickness of said outer layers x; and
a represents an integer greater than or equal to 0, connected to the number of alternated layers N such that a=(N−3)/2;
it being understood that:
preferably, x is a different thickness from y;
when several layers are of thickness x, this means that each layer has a thickness x±0 to 15%, preferably±0 to 10%, more preferentially±0 to 5%;
when several layers are of thickness y, this means that each layer has a thickness y±0 to 15%, preferably±0 to 10%, more preferentially±0 to 5%; and
when several layers are of thickness α x, this means that each layer has a thickness α x±0 to 15%, preferably±0 to 10%, more preferentially±0 to 5%.

As mentioned previously, the first and last layers may consist either of material A with a higher refractive index than B, or of material A with a lower refractive index than B.

Preferably, the higher the refractive index, the lower the thickness of the successive layers x or y and vice versa, the lower the index, the higher the thickness of the layers x or y.

Preferably, the thickness x is less than the thickness y.

According to a particular embodiment of the invention, the maximum thickness of each layer of the multilayer material of the invention is 120 nm; more particularly, the maximum thickness of each layer is 100 nm. Preferably, the thickness x is y is between 5 and 60 nm, more preferentially between 10 and 50 nm and even more preferentially between 20 and 40 nm.

According to an advantageous variant of the invention, in the mathematical formula (I), “a” is an integer greater than or equal to 0 and between 0 and 7, (0≤a≤7; thus 3≤N≤17).

More preferentially, “a” is between 0 and 5 (0≤a≤5; thus 3≤N≤13) and even more preferentially “a” is between 0 and 3 ((0≤a≤3; thus 3≤N≤9).).

Preferably, the multilayer material of the invention has a number N of layers of between 3 and 17 as follows:

In the particular case where N=3, the developed mathematical formula (I) becomes:

[x/y/x]

In the particular case where N=5, the developed mathematical formula (I) becomes:

[x/y/αx/y/x]

In the particular case where N=7, the developed mathematical formula (I) becomes:

[x/y/αx/y/αx/y/x]

In the particular case where N=9, the developed mathematical formula (I) becomes:

[x/y/αx/y/αx/y/αx/y/x]

In the particular case where N=11, the developed mathematical formula (I) becomes:

[x/y/αx/y/αx/y/αx/y/αx/y/x]

In the particular case where N=13, the developed mathematical formula (I) becomes:

[x/y/αx/y/αx/y/αx/y/αx/y/αx/y/x]

In the particular case where N=15, the developed mathematical formula (I) becomes:

[x/y/αx/y/αx/y/αx/y/αx/y/αx/y/αx/y/x]

In the particular case where N=17, the developed mathematical formula (I) becomes:

[x/y/αx/y/αx/y/αx/y/αx/y/αx/y/αx/y/αx/y/x]

It being understood that, for each particular case:
preferably, x is a different thickness from y;
when several layers are of thickness x, this means that each layer has a thickness x±0 to 15%, preferably±0 to 10%, more preferentially±0 to 5%;
when several layers are of thickness y, this means that each layer has a thickness y±0 to 15%, preferably±0 to 10%, more preferentially±0 to 5%; and
when several layers are of thickness α x, this means that each layer has a thickness α x±0 to 15%, preferably±0 to 10%, more preferentially±0 to 5%.

According to a preferred form of the invention, the multilayer material of the invention is such that:
the number N of layers of the multilayer material is such that N is equal to 3, 5, 7, 9, 13 and 17, and/or
A and B constituting each of the alternated layers of said multilayer material are pure inorganic materials chosen from anatase titanium dioxide (TiO₂), titanium dioxide notably vacuum-deposited (TiO₂), sol-gel titanium dioxide (TiO₂), silica (SiO₂), vacuum-deposited silica (SiO₂), and/or
two adjacent layers with different refractive indices such that the difference in refractive index between material A and material B is between 0.3 and 2, preferably between 0.4 and 2, more preferentially between 0.4 and 1.8, even more preferentially between 0.6 and 1.5 or even more preferably between 0.7 and 1.3; and/or
the thicknesses of each of the layers of material A and of material B are less than 100 nm; and
the thickness of each layer obeys the mathematical formula (I) as defined previously.

According to a first embodiment of this preferred form of the invention, the outer layer is a layer with a lower refractive index than the adjacent layer.

According to a second embodiment of this preferred form of the invention, the outer layer has a higher refractive index than the adjacent layer.

According to a particular embodiment, the chemical composition and the thickness of the multilayer materials of the invention with N is equal to 3, 5, 7, 9, 13 and 17 layers are mentioned in the table below with thicknesses for each layer less than 100 nm. In these preferred embodiments, the (in)organic compound with a high refractive index, which is in particular inorganic, is TiO₂ and the (in)organic compound with a lower refractive index, which in particular is also inorganic, is SiO2, with respective refractive indices of 2.5 and 1.5 at 440 nm. Preferably, the outer layers of the multilayer materials of the invention consist of (in)organic compounds, in particular inorganic compounds, having the highest refractive index.

According to a particular embodiment of the invention, the multilayer materials include between 3 and 17 layers and are such that:

TABLE 1

| | | Material | | | | | | |
|---|---|---|---|---|---|---|---|---|
| | | Thickness of the layers x, y | | | | | | |
| | | 3 | 5 | 7 | 9 | 13 | 11 | |
| Layers | | layers | layers | layers | layers | layers | layers | |
| 1 | A | | x | x | x | x | x | x |
| 2 | B | | y | y | y | y | y | y |
| 3 | A | | x | αx | αx | αx | αx | αx |
| 4 | B | | | y | y | y | y | y |
| 5 | A | | | x | αx | αx | αx | αx |
| 6 | B | | | | y | y | y | y |
| 7 | A | | | | x | αx | αx | αx |
| 8 | B | | | | | y | y | y |
| 9 | A | | | | | x | αx | αx |
| 10 | B | | | | | | y | y |
| 11 | A | | | | | | αx | αx |
| 12 | B | | | | | | y | y |
| 13 | A | | | | | | x | αx |
| 14 | B | | | | | | | y |
| 15 | A | | | | | | | αx |
| 16 | B | | | | | | | y |
| 17 | A | | | | | | | x |

it being understood that:
preferably, x has a different thickness from y;
when several layers are of thickness x, this means that each layer has a thickness x±0 to 15%, preferably±0 to 10%, more preferentially±0 to 5%;
when several layers are of thickness y, this means that each layer has a thickness y±0 to 15%, preferably±0 to 10%, more preferentially±0 to 5%; and
when several layers are of thickness α x, this means that each layer has a thickness α x±0 to 15%, preferably±0 to 10%, more preferentially±0 to 5%.

According to a particular embodiment of the invention, the multilayer materials are such that:
A and B are inorganic or organic materials, preferably inorganic materials, of the adjacent layers with A having a higher refractive index than that of material B, the difference in refractive index between the adjacent layers preferably being inclusively between 0.3 and 2, preferably between 0.4 and 2, more preferentially between 0.5 and 1.8, even more preferentially between 0.6 and 1.5 or even more preferentially between 0.7 and 1.3; and
x and y are the thicknesses of the layers of the material with x<y; preferably, they are such that 5 nm≤x≤40 nm and 10 nm≤y≤50 nm, more preferentially 10 nm≤x≤30 nm and 20 nm≤y≤40 nm,
it being understood that the thicknesses of the layers x between each other, αx between each other and y between each other are identical, α being as defined previously. According to a preferred embodiment of the invention, the multilayer materials include between 3 and 17 layers and are such that:

TABLE 2

| | | Material | | | | | |
|---|---|---|---|---|---|---|---|
| | | Thickness of the layers x, y | | | | | |
| | | 3 | 5 | 7 | 9 | 13 | 17 |
| Layers | | layers | layers | layers | layers | layers | layers |
| 1 | TiO₂ | x | x | x | x | x | x |
| 2 | SiO₂ | y | y | y | y | y | y |
| 3 | TiO₂ | x | αx | αx | αx | αx | αx |
| 4 | SiO₂ | | y | y | y | y | y |
| 5 | TiO₂ | | x | αx | αx | αx | αx |
| 6 | SiO₂ | | | y | y | y | y |
| 7 | TiO₂ | | | x | αx | αx | αx |
| 8 | SiO₂ | | | | y | y | y |
| 9 | TiO₂ | | | | x | αx | αx |
| 10 | SiO₂ | | | | | y | y |
| 11 | TiO₂ | | | | | αx | αx |
| 12 | SiO₂ | | | | | y | y |
| 13 | TiO₂ | | | | | x | αx |
| 14 | SiO₂ | | | | | | y |
| 15 | TiO₂ | | | | | | αx |
| 16 | SiO₂ | | | | | | y |
| 17 | TiO₂ | | | | | | x |

Multilayer materials in which x and y are such that x<y, and preferably 5 nm≤x≤40 nm and 10 nm≤y≤50 nm and more preferentially 10 nm≤x≤30 nm and 20 nm≤y≤40 nm and x<y,
it being understood that:
preferably, x has a different thickness from y;
the thicknesses of the layers x between each other, αx between each other and y between each other are identical, α being as defined previously;
when several layers are of thickness x, this means that each layer has a thickness x±0 to 15%, preferably±0 to 10%, more preferentially±0 to 5%;
when several layers are of thickness y, this means that each layer has a thickness y±0 to 15%, preferably±0 to 10%, more preferentially±0 to 5%; and
when several layers are of thickness α x, this means that each layer has a thickness α x±0 to 15%, preferably±0 to 10%, more preferentially±0 to 5%.

According to an even more preferred embodiment of the invention, the multilayer materials include between 3 and 17 layers and are such that:

TABLE 3

| | | Material | | | | | |
|---|---|---|---|---|---|---|---|
| | | Thickness of the layers (nm) | | | | | |
| | | 3 | 5 | 7 | 9 | 13 | 17 |
| Layers | | layers | layers | layers | layers | layers | layers |
| 1 | TiO₂ | 21 | 21 | 21 | 21 | 21 | 21 |
| 2 | SiO₂ | 37 | 37 | 37 | 37 | 37 | 37 |
| 3 | TiO₂ | 21 | 42 | 42 | 42 | 42 | 42 |
| 4 | SiO₂ | | 37 | 37 | 37 | 37 | 37 |
| 5 | TiO₂ | | 21 | 42 | 42 | 42 | 42 |
| 6 | SiO₂ | | | 37 | 37 | 37 | 37 |
| 7 | TiO₂ | | | 21 | 42 | 42 | 42 |
| 8 | SiO₂ | | | | 37 | 37 | 37 |
| 9 | TiO₂ | | | | 21 | 42 | 42 |
| 10 | SiO₂ | | | | | 37 | 37 |
| 11 | TiO₂ | | | | | 42 | 42 |
| 12 | SiO₂ | | | | | 37 | 37 |
| 13 | TiO₂ | | | | | 21 | 42 |
| 14 | SiO₂ | | | | | | 37 |
| 15 | TiO₂ | | | | | | 42 |
| 16 | SiO₂ | | | | | | 37 |
| 17 | TiO₂ | | | | | | 21 |

it being understood that:
when several layers are of thickness of 21 nm, this means that each layer has a thickness of 21 nm±0 to 3.15 nm, preferably 21 nm±0 to 2.1 nm, more preferentially 21 nm±0 to 1.05 nm;
when several layers are of thickness of 37 nm, this means that each layer has a thickness of 37 nm±0 to 5.55 nm, preferably 37 nm±0 to 3.7 nm, more preferentially 37 nm±0 to 1.85 nm; and
when several layers are of thickness of 42 nm, this means that each layer has a thickness of 42 nm±0 to 6.3 nm, preferably 42 nm±0 to 4.2 nm, more preferentially 42 nm±0 to 2.1 nm.

According to another particular embodiment of the invention, the multilayer materials include between 3 and 17 layers and are such that:

TABLE 4

| | | Material | | | | | |
|---|---|---|---|---|---|---|---|
| | | Thickness of the layers x, y | | | | | |
| | | 3 | 5 | 7 | 9 | 13 | 17 |
| Layers | | layers | layers | layers | layers | layers | layers |
| 1 | B | x | x | x | x | x | x |
| 2 | A | y | y | y | y | y | y |
| 3 | B | x | αx | αx | αx | αx | αx |
| 4 | A | | y | y | y | y | y |
| 5 | B | | x | αx | αx | αx | αx |
| 6 | A | | | y | y | y | y |
| 7 | B | | | x | αx | αx | αx |
| 8 | A | | | | y | y | y |
| 9 | B | | | | x | αx | αx |
| 10 | A | | | | | y | y |
| 11 | B | | | | | αx | αx |
| 12 | A | | | | | y | y |
| 13 | B | | | | | x | αx |
| 14 | A | | | | | | y |
| 15 | B | | | | | | αx |
| 16 | A | | | | | | y |
| 17 | B | | | | | | x |

Multilayer Materials in which:
A and B are inorganic or organic materials, preferably inorganic materials, of the adjacent layers with A having a higher refractive index than that of B, the difference in refractive index between the adjacent layers preferably being inclusively between 0.3 and 2, preferably between 0.4 and 2, more preferentially between 0.5 and 1.8, even more preferentially between 0.6 and 1.5 or even more preferentially between 0.7 and 1.3; and
x and y are the thicknesses of the layers of the material such that x<y, preferably 41 nm≤x≤200 nm and 51 nm≤y≤250 nm and x<y, more preferentially 80 nm≤x≤120 nm and 90 nm≤y≤130 nm,
it being understood that:
preferably, x is a different thickness from y; the thicknesses of layers x between each other, α x between each other and y between each other are identical, α being as defined previously;
when several layers are of thickness x, this means that each layer has a thickness x±0 to 15%, preferably±0 to 10%, more preferentially±0 to 5%;
when several layers are of thickness y, this means that each layer has a thickness y±0 to 15%, preferably±0 to 10%, more preferentially±0 to 5%; and
when several layers are of thickness αx, this means that each layer has a thickness αx ±0 to 15%, preferably±0 to 10%, more preferentially±0 to 5%.

According to a preferred embodiment of the invention, the multilayer materials include between 3 and 17 layers and are such that:

TABLE 5

| | | Material | | | | | |
|---|---|---|---|---|---|---|---|
| | | Thickness of the layers x, y | | | | | |
| | | 3 | 5 | 7 | 9 | 13 | 17 |
| Layers | | layers | layers | layers | layers | layers | layers |
| 1 | SiO₂ | x | x | x | x | x | x |
| 2 | TiO₂ | y | y | y | y | y | y |
| 3 | SiO₂ | x | αx | αx | αx | αx | αx |
| 4 | TiO₂ | | y | y | y | y | y |
| 5 | SiO₂ | | x | αx | αx | αx | αx |
| 6 | TiO₂ | | | y | y | y | y |
| 7 | SiO₂ | | | x | αx | αx | αx |
| 8 | TiO₂ | | | | y | y | y |
| 9 | SiO₂ | | | | x | αx | αx |
| 10 | TiO₂ | | | | | y | y |
| 11 | SiO₂ | | | | | αx | αx |
| 12 | TiO₂ | | | | | y | y |
| 13 | SiO₂ | | | | | x | αx |
| 14 | TiO₂ | | | | | | y |
| 15 | SiO₂ | | | | | | αx |
| 16 | TiO₂ | | | | | | y |
| 17 | SiO₂ | | | | | | x |

Multilayer materials in which x and y are such that x<y, and preferentially 41 nm≤x≤200 nm and 51 nm≤y≤250 nm and x<y, more preferentially 80 nm≤x≤120 nm and 90 nm≤y≤130 nm, α being as defined previously;
it being understood that:
preferably, x is a different thickness from y; when several layers are of thickness x, this means that each layer has a thickness x±0 to 15%, preferably±0 to 10%, more preferentially±0 to 5%;
when several layers are of thickness y, this means that each layer has a thickness y±0 to 15%, preferably±0 to 10%, more preferentially±0 to 5%; and
when several layers are of thickness αx, this means that each layer has a thickness αx ±0 to 15%, preferably±0 to 10%, more preferentially±0 to 5%.

According to an even more preferred embodiment of the invention, the multilayer materials include between 3 and 17 layers and are such that:

TABLE 6

| | | Material | | | | | |
|---|---|---|---|---|---|---|---|
| | | Thickness of the layers (nm) | | | | | |
| | | 3 | 5 | 7 | 9 | 13 | 11 |
| Layers | | layers | layers | layers | layers | layers | layers |
| 1 | SiO₂ | 92 | 92 | 92 | 92 | 92 | 92 |
| 2 | TiO₂ | 105 | 105 | 105 | 105 | 105 | 105 |
| 3 | SiO₂ | 92 | 184 | 184 | 184 | 184 | 184 |
| 4 | TiO₂ | | 105 | 105 | 105 | 105 | 105 |
| 5 | SiO₂ | | 92 | 184 | 184 | 184 | 184 |
| 6 | TiO₂ | | | 105 | 105 | 105 | 105 |
| 7 | SiO₂ | | | 92 | 184 | 184 | 184 |
| 8 | TiO₂ | | | | 105 | 105 | 105 |
| 9 | SiO₂ | | | | 92 | 184 | 184 |
| 10 | TiO₂ | | | | | 105 | 105 |
| 11 | SiO₂ | | | | | 184 | 184 |
| 12 | TiO₂ | | | | | 105 | 105 |
| 13 | SiO₂ | | | | | 92 | 184 |
| 14 | TiO₂ | | | | | | 105 |
| 15 | SiO₂ | | | | | | 184 |
| 16 | TiO₂ | | | | | | 105 |
| 17 | SiO₂ | | | | | | 92 |

it being understood that:
when several layers are of thickness of 105 nm, this means that each layer has a thickness of 105 nm±0 to 15.75 nm, preferably 105 nm±0 to 10.5 nm, more preferentially 105 nm±0 to 5.25 nm;
when several layers are of thickness of 92 nm, this means that each layer has a thickness of 92 nm±0 to 13.8 nm, preferably 92 nm±0 to 9.2 nm, more preferentially 92 nm±0 to 4.6 nm; and
when several layers are of thickness of 184 nm, this means that each layer has a thickness of 184 nm±0 to 27.6 nm, preferably 184 nm±0 to 18.4 nm, more preferentially 184 nm±0 to 9.2 nm.

In these embodiments, the UV filtration, in particular in the UVA and long UVA range, and also the satisfactory transparency in the visible range are notably obtained with the use of TiO₂ and SiO₂.

### Process for Preparing the Multilayer Materials

The invention also relates to a process for preparing the multilayer materials of the invention.

Before performing this process,
the (in)organic materials A and B, and preferably inorganic materials, which will constitute the N alternated layers of materials A and B are selected such that the difference in refractive index between material A and material B is between 0.3 and 2, preferably between 0.4 and 2, more preferentially between 0.4 and 1.8, even more preferentially between 0.6 and 1.5 or even more preferably between 0.7 and 1.3;
and
the thickness of the layers is optionally modeled so that the multilayer material obtained has the desired optical properties such as low transmittance in the UV range and high transmittance in the visible range, with a filtration front that is as narrow as possible, characterized by a slope of greater than 2.5. 10⁻³ nm⁻¹, preferably greater than 3. 10⁻³ nm⁻¹, more preferentially greater than 4. 10⁻³ nm⁻¹
   Relationship between the Refractive Index and the Thicknesses of the Layers of the Material:

The relationships between the refractive indices of materials A and B used and the thicknesses of the layers of each of these materials define the “cut-off position” of the transition profile of the transmission between the UVA wavelength range (320 nm to 400 nm) and the visible range (400 nm to 780 nm).

It is possible to model the thickness of the layers to optimize the optical properties.

The calculations linking the thicknesses and the refractive index of the (in)organic compounds A and B constituting the layers of the multilayer material of the invention with the optical properties (transmission, reflection, absorption) may notably be performed via the “Transfer Matrix Method” or using “FDTD algorithms”.

### Transfer Matrix Method:

[1] P. Yeh, Optical Waves in Layered Media (Wiley, New York, 1988)
[2] Z. Knittl, Optics of Thin Films: An Optical Multilayer Theory (Wiley, London, 1976).
[3] O. S. Heavens, Optical Properties of Thin Films (Dover, New York, 1965)
[4] M. Claudia Troparevsky et. al., Transfer-matrix formalism for the calculation of optical response in multilayer systems: from coherent to incoherent interference. Optics Express vol. 18, Issue 24, pp. 24715-24721 (2010)

### FDTD:

[1] Dennis M. Sullivan, Electromagnetic simulation using the FDTD method. New York: IEEE Press Series, (2000).
[2] Allen Taflove, Computational Electromagnetics: The Finite-Difference Time-Domain Method. Boston: Artech House, (2005).
[3] Stephen D. Gedney, Introduction to the Finite-Difference Time-Domain (FDTD) Method for Electromagnetics. Morgan & Claypool publishers, (2011).

Or via other “open source” algorithms that are available, for example, at the address

https://fr.mathworks.com/matlabcentral/fileexchange/47637-transmittance-and-reflectance-spectra-of-multilayered-dielectric-stack-using-transfer-transfer-transfer-mansx-method.
Commercial Algorithms may also be Used, for Instance:
http://www.lighttec.fr/optical-design-software/tfcalc/
https://www.lumerical.com/products/fdtd-solutions/

According to a particular embodiment of the invention, the iterative calculations for optimizing the “cut-off” position are performed via optimization algorithms such as a “particle swarm algorithm” or “genetic algorithms” in combination with or without the abovementioned algorithms.

References for these Algorithms:

### Particle Swarm Algorithm:

[1] Kennedy, J., and R. Eberhart. “Particle Swarm Optimization.” Proceedings of the IEEE International Conference on Neural Networks. Perth, Australia, 1995, pp. 1942-1945.
[2] Mezura-Montes, E., and C. A. Coello Coello. “Constraint-handling in nature-inspired numerical optimization: Past, present and future.” Swarm and Evolutionary Computation. 2011, pp. 173-194.
[3] Pedersen, M. E. “Good Parameters for Particle Swarm Optimization.” Luxembourg: Hvass Laboratories, 2010.

### Genetic Algorithm:

[1] Goldberg, David E., Genetic Algorithms in Search, Optimization & Machine Learning, Addison-Wesley, 1989.
[2] A. R. Conn, N. I. M. Gould, and Ph. L. Toint. “A Globally Convergent Augmented Lagrangian Algorithm for Optimization with General Constraints and Simple Bounds”, SIAM Journal on Numerical Analysis, Volume 28, Number 2, pages 545-572,1991.
[3] A. R. Conn, N. I. M. Gould, and Ph. L. Toint. “A Globally Convergent Augmented Lagrangian Barrier Algorithm for Optimization with General Inequality Constraints and Simple Bounds”, Mathematics of Computation, Volume 66, Number 217, pages 261-288, 1997.

During the modeling, the optimization on the thicknesses of the various layers x and y for N<9 is preferably performed on a material with N′ layers comprising at least 9 layers, more preferably at least 13 layers and even more preferably at least 15 layers.

According to a particular embodiment of the invention, the optimization is performed for a material comprising N layers, where N is less than 9. Its design will be produced by the iterative methods mentioned previously according to the following principle:
1. modeling a multilayer material with N′ layers, where N′>N; N′ is defined at least equal to 9, more preferably equal to 13 and even more preferably equal to 15;
2. iteration for optimization of the values x and y for N′

N′=[x/y/(αx/y)_{a′}/x]
a′ is defined as an integer greater than or equal to 0, α is as defined previously;
3. using the values of x, αx, and y obtained during the design of N′ for the design of the multilayer material with N layers without subsequent optimization;

N=[x/y/(αx/y)_{z}/x]
a is defined as an integer greater than or equal to 0 and a′>a, α is as defined previously.

By following these construction instructions for N<9, the cut-off of the protecting agent with N layers may possibly fall outside the cut-off range [380 nm-420 nm]; in these cases, combination with either a particular mode of preparation of multilayer materials, or with specific application modes, or a combination of the two, make it possible to ensure a cut-off within the range.

The iterative approach may also be combined with the general knowledge of a person skilled in the art regarding multilayer materials and also regarding the manufacturing processes used and known in the field by a person skilled in the art.

One subject of the invention is the process for preparing the multilayer materials as defined previously, comprising the following steps:
1. preparing a substrate and optionally applying to the substrate at least one nonstick layer, also known as a sacrificial layer, onto said substrate;
2. depositing an odd number N of alternated layers of materials A and B consisting of (in)organic compounds of high and lower refractive index, or of low and higher refractive index, onto the substrate optionally coated with sacrificial layer;
3. detaching the multilayer material from the substrate optionally coated with sacrificial layer;
4. if necessary, adjusting the size of the multilayer material to obtain multilayer material particles; and
5. optionally performing a post-treatment optionally followed by a (re)adjustment.

The term “substrate” means a support for applying the various successive layers of (in)organic materials A and B with different refractive indices; this substrate may be in the form of metal plates, sheets, wovens or nonwovens, or consist of glass, of natural or non-natural polymeric compound such as plastics, nonconductors or (semi)conductors. This substrate may be flat or non-flat, rounded or spherical, preferably flat.

According to one embodiment of the invention, the multilayer material having an odd number N of layers contains also a substrate.

According to a particular embodiment of the invention, the substrate consists of an inorganic compound such as glass, silicon or quartz, of metal such as aluminum or of an organic compound preferably chosen from the following organic polymers: poly(methyl methacrylate) (PMMA), poly(ethylene terephthalate) (ET), polypropylene (PP), polyethylene (PE), polyvinyl chloride (PVC), polyimide (PI), nylons, celluloses and derivatives thereof such as paper, or cotton. According to a particular embodiment, the substrate is inorganic such as glass or quartz, preferably glass.

The multilayer materials of the invention may also be manufactured on metal substrates, semiconductors or metal oxides.

Preferably, the process for manufacturing the multilayer materials of the invention comprises the following steps: 1) providing a substrate, 2) depositing a sacrificial layer onto the substrate, and then 3) successive deposition of an odd number of alternated layers of (in)organic materials A and B onto the sacrificial layer, and then 4) the sacrificial layer is selectively removed, in particular by exposure to a chemical solution, and 5) the multilayer material thus obtained is optionally subjected to a treatment to adjust its size and/or to a post-treatment.

According to another embodiment of the invention, the multilayer material having an odd number N of layers is free of substrate.

According to a particular embodiment of the invention, the process for preparing the multilayer materials involves a nonstick layer also known as a sacrificial layer.

If the process involves the application of a sacrificial or nonstick layer, then the substrate must be inert with respect to said sacrificial or nonstick layer.

In particular, the compounds that may be used in the sacrificial layer are chosen from the following polymers: i) acenaphthylene/MMA polymer; ii) acenaphthylene/styrene/acrylic polymer; iii) acrylic/butadiene/styrene polymer; iv) (acrylonitrile/butadiene/styrene)amides polymer; v) acrylimide/acrylic acid polymer; vi) (low molecular weight) acetylene polymer; vii) acrylic polymer; viii) acrylonitrile/butadiene (rubber) polymer; viii) alkyd resins; ix) alkyl resins preferably of (C₁-C₈)alkyl; x) alkylene glycol polymer preferably of (C₁-C₈)alkylene; xi) amide/imide polymer; xii) acrylonitrile polymer; xiii) acrylic acid polymer; xiv) amylose propylate polymer; xv) amylose acetate polymer; xvi) amylose butylate polymer; xvii) acrylonitrile/styrene polymer; xviii) 1-butene polymer; xix) butyl rubber; xx) butyl methacrylate polymer; xxi) butylene terephthalate polymer; xxii) butadiene/acrylic polymer; xxiii) acid/acrylonitrile butyl isocyanate polymer; xxiv) cellulose acetate polymer; xxv) cellulose nitrate polymer; xxvi) halogenated, notably chlorinated, polyethylene polymer (chloroprene); xxvii) caprolactam polymer; xxviii) carbonate polymer; xxix) carboxylated polybutadiene polymer; xxx) carboxy(C₁-C₆)alkylcellulose polymer, preferably carboxymethylcellulose polymer; xxxi) cis-trans isoprene polymer (preferably cis-isoprene); xxxii) cellulose trinitrate polymer; xxxiii) dextran polymer; xxxiv) dialkyl phthalate polymer, preferably di(C₁-C₆)alkyl phthalate polymer; xxxv) dimethylsiloxane polymer; xxxvi) dodecyl acrylate polymer; xxxvii) dioxalane polymer; xxxvii) (C₂-C₆)alkylene oxide polymer, preference ethylene oxide polymer; xxxviii) polyethers; xxxix) epichlorohydrin polymer; xxxx) epoxy resins; xxxxi) (C₁-C₆)alkyl acrylate, preferably ethyl acrylate polymer; xxxxii) (C₂-C₆)alkylene/(C₁-C₆)alkylcarbonyl(C₂-C₆)alkylenoxy polymer, preference ethylene/vinyl acetate (EVA) polymer; xxxxiii) (C₂-C₆)alkylene/(C₂-C₆)alkylene polymer, preferably ethylene/propylene polymer; xxxxiv) (C₂-C₆)alkylene terephthalate polymer, preferably polyethylene terephthalate (PET) polymer; xxxxv) (C₂-C₆)alkylene/(C₂-C₆)alkenoic acid polymer or salts thereof with an alkaline agent or with alkali metals or alkaline-earth metals, and (C₁-C₆)alkyl esters thereof, preferably ethylene/acrylic acid polymer or salts thereof with an alkaline agent or with alkali metals or alkaline-earth metals and the (C₁-C₆)alkyl esters thereof; xxxxvi) (C₂-C₆)alkylene/(C₂-C₆)alkenoic acid/(C₂-C₆)alkenylcarbonyloxy(C₁-C₆)alkyl polymer, preferably ethylene/methylacrylate polymer; xxxxvii) ethylene/1-hexane polymer; xxxxviii) polyesters; xxxxix) fatty acid polymer; L) furfuryl alcohol polymer; Li) gelatin polymer; Lii) glyceride polymer; Liii) glycol ester/glycerol polymer; Liv) polyglycols; Lv) polyisoprene; Lvi) polyisobutylene; Lvii) polyisocyanates; Lviii) polyimides; Lix) imic acid polymer; Lx) aryl(C₂-C₆)alkenyl polymer, preferably isopropylidene-1,4-phenylene polymer; Lxi) lignin sulfonates; Lxii) lipid polymer; Lxiii) melamines; Lxiv) (C₂-C₆)alkenoic acid polymer or salts thereof with an alkaline agent or with alkali metals or alkaline-earth metals and the (C₁-C₆)alkyl esters thereof, preferably methyl methacrylate polymer; Lxv) polymethylacrylates; Lxvi) (C₂-C₆)alkenoic acid polymer or salts thereof with an alkaline agent or with alkali metals or alkaline-earth metals and the (C₁-C₆)alkyl esters thereof/aryl(C₂-C₆)alkenyl, preferably methyl methacrylate/styrene polymer; Lxvii) methylpentene polymer; Lxviii) oxycarbonylarylene polymer, preferably oxycarbonyloxy-1,4-phenylene polymer; Lxix) oxy(C₁-C₆)alkylene polymer, preferably polyoxypropylene or polyoxymethylene; Lxxi) polymer of (C₂-C₆)alkenoic acid ester and of (C₈-C₂₀)alkanol, preferentially octadecyl methacrylate polymer; Lxxii) (C₈-C₂₀)alkenyl polymer; Lxxiii) oxymaleoyloxy(C₁-C₈)alkylene polymer, preferably oxymaleoyloxhexamethylene polymer; Lxxiv) oxysuccinyloxy(C₁-C₈)alkylene polymer, preferably oxysuccinyloxhexamethylene polymer; Lxxv) polyols; Lxxvi) hydroxyaryl polymers, preferably phenolic polymer; Lxxvi) phenol-formaldehyde resins; Lxxvii)oxyarylene polymer, preferably polyphenylene oxide; Lxxviii) polypropylene; Lxxix) poly(C₁-C₆)alkylene oxide, preferably polypropylene oxide; Lxxx) propylene/1-butene polymer; Lxxxi) polyvinyl acetate; Lxxxii) polyvinyl alcohol (PVA); Lxxxiii) polymer of vinyl butyral; Lxxxiv) polymer of vinyl halide, notably vinyl chloride, or vinyl fluoride polymer; Lxxxv) vinyl methyl ether polymer; Lxxxvi) vinyl halide/vinyl polymer, notably vinyl chloride/vinyl polymer; Lxxxvii) acetate/maleic acid/vinyl alcohol/vinyl acetate polymer; Lxxxv) polyvinyl esters; Lxxxvi) polyvinylpyrrolidone/vinyl acetate; Lxxxvii) vinyl acetate/ethylene polymer; Lxxxix) vinyl acetate/ethylene/acrylate polymer; xC) vinyl halide polymer, notably vinyl bromide polymer; xCi) ferrocene vinyl polymer; xCii) vinyl carbazole polymer; xCiii) vinyl formaldehyde polymer; xCiv) cellulose propionates; and xCv) vinyl resins.

In particular, the sacrificial layer consists of organic compounds chosen from soluble polymers such as vinyl resins (for example poly(vinyl acetate), polyvinyl alcohol (PVA), polyvinylpyrrolidone (PVP), acrylic and methacrylic resins (polyacrylic acid (PAA), polymethacrylic acid (PMAA), polyacrylamide), polyethylene glycols (PEG), cellulose and derivatives thereof, (poly-oligo-mono-)saccharides, and organic salts.

The sacrificial layer may also consist of inorganic compounds, metals and/or semiconductors such as aluminum, aluminum gallium arsenide, dialuminum trioxide/alumina/sapphire, antimony, bismuth, brass, bronze, carbon, chromium, cobalt, copper, gallium arsenide, germanium, indium, indium gallium arsenide, indium gallium phosphide, indium phosphide, indium phosphide oxide oxides, iridium, iron, lead, magnesium, molybdenum, nickel, niobium, tin, titanium, tungsten, vanadium, zinc, similar alloys, and also inorganic salts.

According to another variant, the preparation process consists in depositing a sacrificial layer onto the substrate, and then in alternately depositing an odd number N of layers of (in)organic compounds A and B with a high refractive index and a lower refractive index onto said nonstick or sacrificial layer.

The deposition step may be performed via well-known processes for depositing successive thin films. These deposition processes may include, without being limited thereto, vapor deposition processes such as chemical vapor deposition (CVD) or physical vapor deposition (PVD), or wet chemical processes such as precipitation or sol-gel condensation, or wet-route coating using a roll-to-roll process, deposition using a roller, spin coating, and dip coating. The majority of these processes are partially described in the book “Special Effect Pigments”, Gerhard Pfaff, ISBN 9783866309050.

The separation or delamination of the multilayer material from the substrate or from the sacrificial layer may be performed by dissolution, thermal decomposition, mechanical action, chemical attack, irradiation or a combination of these operations. Processes for detaching the multilayer material from the substrate or from the sacrificial layer may be found in US 2012/0256333 A1 “Process for manufacturing an autonomous multilayer thin film”.

According to one embodiment of the preparation process, the sacrificial layer and the various layers of (in)organic materials A and B with a high refractive index and with a lower refractive index are exposed to an aqueous chemical solution which is either an alkaline attack agent, i.e. an alkaline solution (pH>7), or an acidic attack solution, i.e. an acidic solution (pH<7), or an aqueous or organic solvent. Exposure of the substrate, of the sacrificial layer and of the multilayer material of the invention to an alkaline solution or to an acidic solution or to a solvent makes it possible to dissolve the sacrificial layer, thus releasing the multilayer material of the invention from the substrate.

According to another variant, the chemical solution is an organic or mineral solvent, which dissolves the sacrificial layer, thus releasing the multilayer material from the substrate.

Once released from the substrate, the multilayer material of the invention is then “autonomous”, i.e. free of substrate and of sacrificial or nonstick layer.

According to a particular embodiment of the process of the invention, the sacrificial layer is a metallic and/or semiconducting layer such as aluminum deposited notably using a vacuum deposition technique. The compound that is useful for destroying said metallic sacrificial layer is then an alkaline solution which will specifically react with said sacrificial layer so as to detach the substrate from the multilayer material of the invention without disrupting the UV-screening optical properties. To make the solution alkaline, mention may be made of the use of alkaline agents notably chosen from alkali metal or alkaline-earth metal hydroxides such as sodium hydroxide.

According to another particular embodiment of the process of the invention, the sacrificial layer is organic, and more particularly said layer is an organic polymer.

According to this embodiment, the organic sacrificial layer is separated from the multilayer material of the invention with a solvent or with an alkaline solution or with an acidic solution.

As examples of organic sacrificial layers, mention may be made of the following compounds for which the nature of the solvent or of the alkaline solution or of the acidic solution to be used for separating said sacrificial layer from the multilayer material of the invention is specified:
i) acenaphthylene/MMA polymer organic solvent to dissolve the sacrificial layer (solv.): tetrahydrofuran (THF), dimethylformamide (DMF); ii) acenaphthylene/styrene/acrylic polymer: solv. THF, DMF; iii) acrylic/butadiene/styrene polymer: solv. THF, DMF, dimethyl sulfoxide (DMSO); iv) (acrylonitrile/butadiene/styrene)amides polymer: solv. DMF; v) acrylimide/acrylic acid polymer: solv. H₂O+alkali metal acetate+alkali metal phosphate, DMSO; vi) acetylene polymer (low molecular weight): solv. toluene, 1,2,4-trichlorobenzene (TCB); vii) acrylic polymer: solv. toluene, THF, DMF, DMSO; viii) acrylonitrile/butadiene polymer (rubber): solv. toluene, DMF, TCB; viii) alkyd resins: solv. toluene, THF, chloroform, dimethylacetamide (DMAC); ix) alkyl resins: solv. THF, chloroform; x) alkylene glycol polymer: solv. ortho-dichlorobenzene (ODCB), toluene, THF, chloroform; xi) amide/imide polymer: solv. DMF, DMAC, DMSO, DMF+LiBr; xii) acrylonitrile polymer: solv. DMF; xiii) acrylic acid polymer: solv. H₂O+alkali metal, alkaline-earth metal or ammonium acetate salt (preferably 0.05 M)+polar protic organic solvent such as methanol (preferably 2% by weight), at a pH preferably between 7 and 8, such as 7.2 (which may be adjusted with an alkaline agent such as NH₄OH); xiv) amylose propylate polymer: solv. THF; xv) amylose acetate polymer: solv. THF; xvi) amylose butylate polymer: solv. THF; xvii) acrylonitrile/styrene polymer: solv. THF; xviii) 1-butene: solv. ODCB, toluene, TCB; xix) butyl rubber: solv. ODCB, toluene, TCB; xx) butyl methacrylate polymer: solv. DMF; xxi) butylene terephthalate polymer: solv. m-cresol; xxii) butadiene/acrylic polymer: solv. toluene, DMF; xxiii) acid/acrylonitrile butyl isocyanate polymer: solv. THF; xxiv) cellulose acetate polymer: solv. THF, DMF; xxv) cellulose nitrate polymer: solv. THF; xxvi) chlorinated polyethylene polymer (chloroprene): solv. TCB; xxvii) caprolactam polymer: solv. m-cresol, HFIP; xxviii) carbonate polymer: ODCB, THF, TCB; xxix) carboxylated polybutadiene polymer; solv. THF; xxx) carboxymethylcellulose polymer: solv. H₂O, DMF; xxxi) isoprene polymer (preferably cis-isoprene): solv. THF; xxxii) cellulose trinitrate polymer: solv. THF; xxxiii) dextran polymer: solv. H₂O, DMSO; xxxiv) dialkyl phthalate polymer: solv. ODCB, toluene, chloroform, TCB; xxxv) dimethylsiloxane polymer: solv. ODCB, toluene, TCB, chloroform; xxxvi) dodecyl acrylate polymer: solv. THF; xxxvii) dioxalane polymer: solv. THF; xxxvii) ethylene oxide polymer: solv. THF, DMF, H₂O, TCB;) (xxviii) polyethers: solv. toluene, THF, DMF; xxxix) epichlorohydrin polymer: solv. TCB; xxxx) epoxy resins: solv. toluene, THF, chloroform; xxxxi) ethyl acrylate polymer: solv. ODCB, toluene, DMF, m-cresol; xxxxii) ethylene/vinyl acetate (EVA) polymer: solv. TCB; xxxxiii) ethylene/propylene polymer: solv. ODCB, TCB; xxxxiv) polyethylene terephthalate (PET): solv. m-cresol, HFIP; xxxxv) ethylene/acrylic acid polymer (NA+form): solv. TCB; xxxxvi) ethylene/methylacrylate polymer: solv. TCB; xxxxvii) ethylene/1-hexane polymer: solv. TCB; xxxxviii) polyesters: solv. m-cresol, HFIP, TCB, toluene; xxxxix) fatty acid polymer, solv. ODCB, THF, chloroform, TCB; L) furfuryl alcohol polymer: solv. ODCB, THF, chloroform, TCB; Li) gelatin polymer: solv. H₂O, DMSO; Lii) glyceride polymer: solv. ODCB, THF, TCB; Liii) glycol/glycerol polyesters: solv. DMF, DMF +0.005% LiBr; Liv) polyglycols: solv. ODCB, toluene, THF, DMF, TCB; Lv) polyisoprene: solv. toluene, TCB; Lvi) polyisobutylene: solv. toluene, THF; Lvii) polyisocyanates: solv. toluene, THF, DMF, chloroform; Lviii) polyimides: solv. DMAC, DMF; Lix) imic acid polymer: solv. NMP; Lx) isopropylidene-1,4-phenylene polymer: solv. THF; Lxi) lignin sulfonates: solv. H₂O; Lxii) lipid polymer: solv. methylene chloride, THF; Lxiii) melamines: solv. HFIP, m-cresol, TFA, TCB; Lxiv) methyl methacrylate polymer: solv. toluene, THF, DMF, m-cresol, DMAC; Lxv) polymethylacrylates: solv. TCB, DMF, THF; Lxvi) methyl methacrylate/styrene polymer: solv. ODCB, toluene, THF, chloroform; Lxvii) methylpentene polymer: solv. TCB; Lxviii) oxycarbonyloxy-1,4-phenylene polymer: solv. THF; Lxix) polyoxypropylene: solv. THF; Lxx) polyoxymethylene: solv. DMAC; Lxxi) polyoctadecyl methacrylate: solv. DMF, hot DMSO (140° C.); Lxxii) octadecylvinyl polymer: solv. THF; Lxxiii) oxymaleoyloxhexamethyene polymer: solv. THF; Lxxiv) oxysuccinyloxy-hexamethylene polymer: solv. THF; Lxxv) polyols: solv. THF, DMF; Lxxvi) phenolic polymers (notably novolacs): solv. THF, chloroform; Lxxvi) phenol-formaldehyde resins: solv. THF, TCB; Lxxvii) polyphenylene oxide: solv. TCB; Lxxviii) polypropylene: solv. ODCB, TCB; Lxxvii) polypropylene oxide: solv. THF, TCB; Lxxx) propylene/1-butene polymer: solv. ODCB, TCB; Lxxvii) polyvinyl acetate: solv. ODCB, THF, DMF; Lxxxii) polyvinyl alcohol: solv. H₂O, preferably hot (50 to 80° C.), DMF, DMSO; Lxxxiii) polymer of vinyl butyral: solv. THF, DMF; Lxxxiv) polymer of vinyl halide, notably of vinyl chloride: solv; toluene, THF; polymer of vinyl fluoride; solv. DMF; Lxxxv) vinyl methyl ether polymer: solv. THF, DMF; Lxxxvi) vinyl halide/vinyl polymer, notably vinyl chloride/vinyl polymer: solv. DMF; Lxxxvii) acetate/maleic acid/vinyl alcohol/vinyl acetate polymer: solv. DMF, DMSO; Lxxxv) polyvinyl esters: solv. DMF, THF; Lxxxvi) polyvinylpyrrolidone/vinyl acetate: solv. DMF; Lxxxvii) vinyl acetate/ethylene polymer: solv. DMF; Lxxxix) vinyl acetate/ethylene/acrylate polymer: solv. DMF; xC) polymer of vinyl halide, notably vinyl bromide: solv. THF; xCi) vinyl ferrocene polymer: solv. THF; xCii) vinylcarbazole polymer: solv. THF; xCiii) vinyl formaldehyde polymer: solv. THF; xCiv) cellulose propionate: solv. alcohols and ketones, in particular C₁-C₆ alcohols and C₁-C₆ dialkyl ketone; and xCv) vinyl resins, solv. acetone or ethanol.

It is also seen that the removal of the nonstick or sacrificial layer using a chemical solution to release the multilayer material without substrate does not affect the color or the optical properties of said multilayer material. For example, the visual color, the absorption properties, the reflection properties, etc. of the multilayer material remain identical or equivalent to what they were before the removal of the sacrificial layer.

According to a particular embodiment for preparation of multilayer materials including N layers, where N is less than 17, more preferentially N is less than 13 and even more preferentially N is less than 9, a post-treatment is performed after the delamination step 3 and/or after the size adjustment step 4.

This post-treatment consists in stacking at least two particles of multilayer materials of (in)organic compounds containing N layers, preferably in the form of flat particles. This stacking is performed in the alternating axis of the layers x and y.

Mention may Notably be made of the Following Post-Treatment Processes:
thermal processes (drying, sintering, atomization, calcination),
mechanical processes (compression, centrifugation, mechanofusion, granulation)
processes driven by physicochemical methods for self-assembly, for example: pH adjustment, optimization of the solvents (cosolvents), use of additives.
chemical processes such as crosslinking by formation of covalent bonds between the unit multilayer materials in the direction of intercalation of the layers x and y
or a combination of two or more of the processes mentioned.

According to a particular embodiment, the preparation of multilayer materials of the invention involves step 4) which consists in adjusting the size of the multilayer material. This step 4) consists in performing milling and/or screening in order to homogenize the size distribution of the multilayer particles to the desired values.

Milling is performed to obtain particles with a size of less than 1000 μm (D90 by volume), preferentially with a size of less than 700 nm (D90 by volume) and even more preferentially with a size of less than 400 nm (D90 by volume). This size distribution may be determined by using laser scattering granulometry, for example with the Mastersizer 2000 machine from Malvern Instruments Ltd.

Screening is performed to select particles as a function of their size and thus to obtain better size homogeneity of the multilayer materials of the invention. For example, the screening may be performed to select particles with a size of between 20 and 400 μm.

The present invention also relates to a cosmetic use of a multilayer material, as an active ingredient for screening out UV rays.

The present invention also relates to a composition, in particular a cosmetic composition, for topical use intended to be applied to keratin materials, notably human keratin materials, in particular the skin, keratin fibers, in particular the hair, and the nails, comprising at least one multilayer material of the invention as defined previously.

### Compositions of the Invention

The multilayer material may be in dry form (powder, flakes, plates), as a dispersion or as a liquid suspension or as an aerosol. The multilayer material may be used in the form as provided or may be mixed with other ingredients.

One subject of the invention is a composition comprising one or more multilayer materials as defined previously.

The composition of the invention may be in various galenical forms. Thus, the composition of the invention may be in the form of a powder composition (pulverulent) or of a liquid composition, or in the form of a milk, a cream, a paste or an aerosol composition.

The compositions according to the invention are in particular cosmetic compositions, i.e. the multilayer material(s) of the invention are in a cosmetic medium. The term “cosmetic medium” means a medium that is suitable for application to keratin materials, notably human keratin materials such as the skin, said cosmetic medium generally consisting of water or of a mixture of water and of one or more organic solvents or of a mixture of organic solvents. Preferably, the composition comprises water and in a content notably of between 5% and 95% inclusive relative to the total weight of the composition. The term “organic solvent” means an organic substance that is capable of dissolving another substance without chemically modifying it.

### Organic Solvents:

Examples of organic solvents that may be mentioned include lower C₂-C₆ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

The organic solvents are present in proportions preferably inclusively between 0.1% and 40% by weight approximately relative to the total weight of the composition, more preferentially between 1% and 30% by weight approximately and even more particularly inclusively between 5% and 25% by weight relative to the total weight of the composition.

The compositions of the invention may contain a fatty phase and may be in the form of direct or inverse emulsions.

The compositions of the invention contain between 0.1% and 40% of multilayer materials, in particular from 0.5% to 20%, more particularly from 1% to 10% and preferentially 1.5% to 5% by weight relative to the total weight of the composition.

The concentration of multilayer materials in the composition may be adjusted as a function of the number N of layers constituting the multilayer material(s) included in the composition.

The compositions of the invention may be used in single application or in multiple application. When the compositions of the invention are intended for multiple application, the content of multilayer material(s) is generally lower than in compositions intended for single application.

For the purposes of the present invention, the term “single application” means a single application of the composition, this application possibly being repeated several times per day, each application being separated from the next one by one or more hours, or an application once a day, depending on the need.

For the purposes of the present invention, the term “multiple application” means application of the composition repeated several times, in general from 2 to 5 times, each application being separated from the next one by a few seconds to a few minutes. Each multiple application may be repeated several times per day, separated from the next one by one or more hours, or each day, depending on the need.

### Application Process

Said multilayer material of the invention is an agent for protecting against UVA and UVB; it notably improves the overall screening-out of UV while at the same time maintaining good overall transmission in the visible range.

It appears that the multilayer materials of the invention, by virtue i) of their specific designs, ii) of the choice of thickness of each layer, iii) of the chemical composition of organic and/or inorganic compounds, iv) of the choice of organic and/or inorganic compounds with a low and a higher diffraction coefficient, and iv) of the suitable preparation method, and v) of the suitable application method, notably make it possible to afford:
UV-screening properties, in particular in the UVA range (cut-off position λ_{c});
improvement of the cut-off transition by means of a “steep” slope centered on λ_{c}; and
excellent transparency in the visible range (400-780 nm).

The multilayer materials of the invention are used in the cosmetic compositions, in particular for application to keratin materials, notably human keratin materials such as the skin, at a concentration preferably between 0.1% and 40% by weight relative to the total weight of the composition comprising them; more preferentially between 0.5% and 20% by weight relative to the total weight of the composition comprising them.

The concentrations of multilayer materials of the invention may be adjusted as a function of the number N of layers of said material. The composition may be in any presentation form.

The materials of the invention may be applied to the keratin materials either as a single application or as multiple applications. For example, a cosmetic composition comprising at least one multilayer material according to the invention may be applied once.

According to another variant, the application process involves several successive applications on the keratin materials of a cosmetic composition comprising at least one multilayer material according to the invention.

They may also be connected application methods, such as a saturated single application, i.e. the single application of a cosmetic composition with a high concentration of multilayer materials according to the invention, or with multiple applications of cosmetic composition (less concentrated) comprising at least one multilayer material according to the invention. In the case of multiple applications, several successive applications of cosmetic compositions comprising at least one multilayer material of the invention are repeated with or without a delay between the applications.

Another subject of the invention is a process for treating keratin materials, notably human keratin materials such as the skin, by application to said materials of a composition as defined previously, preferably by 1 to 5 successive applications, leaving to dry between the layers, the application(s) being sprayed or otherwise.

According to one embodiment of the invention, the multiple application is performed on the keratin materials with a drying step between the successive applications of the cosmetic compositions comprising at least one multilayer material according to the invention. The drying step between the successive applications of the cosmetic compositions comprising at least one multilayer material according to the invention may take place in the open air or artificially, for example with a hot air drying system such as a hairdryer.

According to a preferred embodiment of the invention, the multilayer material is in particle form.

According to a particular embodiment of the invention, the multilayer material(s) of the invention are incorporated into the cosmetic composition, the multilayer materials of the invention and in particular the particles may be stacked according to specific processes along the alternating axis of the layers x and y before or after the application according to the specific preparation methods and application methods.

The multilayer materials of the invention, and the composition comprising them and the methods for applying the multilayer materials of the invention, make it possible notably to improve the state of dispersion and the coverage of the particles, and to improve the UV-screening properties, and/or the transparency in the visible range and the UV→visible cut-off.

Another subject of the invention is the use of one or more multilayer materials as defined previously, as UVA and UVB screening agent for protecting keratin materials, notably the skin.

The examples that follow serve to illustrate the invention without, however, being limiting in nature.

### EXAMPLES

### Preparation of the Multilayer Materials

Measurement of the UV-screening properties of the multilayer materials of the invention and outside the invention

Comparison between a 5-Layer Material According to the Invention and Outside the Invention

Two 5-layer samples were manufactured via standard methods by vapor deposition (CVD/PVD, S5) on 9×9 cm transparent glass substrates. A thin layer of water-soluble PVA polymer (JP-05® Japan Vam and Poval Co) was applied to the surface of the glass plates as nonstick (sacrificial) layer before the vapor deposition. The multilayer materials were prepared by detachment of the abovementioned films from the glass substrate after immersion in hot water (50° C.) for 6 hours. Once detached, the multilayer materials were recovered by filtration and redispersed in deionized water. The first multilayer material ML1 is according to the invention. The second multilayer material ML2 outside the invention was designed as comparative.

The thicknesses detailed and compositions of each layer are given in the following table:

TABLE 7

| | Chemical | ML1 (invention) | ML2 (outside the invention) |
|---|---|---|---|
| Layers | composition | Layer thickness (nm) | Layer thickness (nm) |
| 1 | TiO₂ | 21 | 32 |
| 2 | SiO₂ | 37 | 34 |
| 3 | TiO₂ | 42 | 67 |
| 4 | SiO₂ | 37 | 38 |
| 5 | TiO₂ | 21 | 22 |

The measurements of transmittance between the 5-layer materials ML1 and ML2 were performed as follows:

### Saturated Application:

A drop of a dispersion of multilayer material at 1.7% by weight in deionized water was deposited onto a quartz substrate. After total evaporation of the water, the transmittance measurement was performed.

### Successive Multiple Applications:

A brush was immersed in the dispersion of multilayer materials (1.7% by weight) and the excess multilayer material was removed, followed by applying a continuous coat to the quartz substrate. After evaporation of the water under room temperature conditions (20° C.), the operation was performed three times with measurement of the transmittance and microscopy in each step in order to see the influence of the surface covering and of the amount of material on the optical properties.

### Application by Spraying:

In order to vary the study on the applications of the multilayer material, coating by spraying was tested. Before applying the material to the substrate, the size of ML1 was reduced by treatment with an Ultra-Turrax® machine for 5 minutes at 15 000 rpm, giving rise to sML1. The size comparison is found in the table below:

TABLE 8

| Sample | D50 μm (volume) |
|---|---|
| ML1 | 107.7 ± 3.15 |
| sML1 | 33.5 ± 0.07 |
| ML2 | 103.1 ± 2.26 |

The particle size distributions were determined by laser scattering using a Malvern Instruments Ltd Master Size 2000 granulometer. This laser scattering particle size analyzer uses a blue light (wavelength of 488.0 μm) and a red light (He-Ne wavelength of 633.8 μm).

### Double-Wavelength and Single-Lens Detection System.

An Ecospray rechargeable micro-sprayer with a disposable gas-pressure tank was used to apply a dispersion sML1 of inorganic compounds onto the substrate. The application was performed on a hot substrate so as to accelerate the evaporation of the water, while maintaining a distance of about 25 cm between the sprayer and the substrate. This procedure was repeated three more times, waiting 5 minutes between each application.

### Optical Performance of the 5-Layer Materials According to the Invention Versus Outside the Invention

The transmittance measurements were taken with a USB4000-UV-VIS spectrophotometer (Ocean Optic) equipped with a reflectance-transmittance integration sphere (Oriel Instruments, model 70491). The transmittance data were recorded on a quartz substrate as foundation; its effect was subtracted by using an identical uncoated quartz as blank in the double beam. The light source was established between 200 and 800 nm, DH-2000-BAL Ocean Optics.

### Transmission Analysis:

TABLE 9

| | (nm) | | | | |
|---|---|---|---|---|---|
| Application | UV | UVB | UVA | Visible | |
| process | 250-400 | 290-320 | 320-400 | 421-700 | 400-500 |
| 1 application ML1 (Invention) | 0.43 | 0.36 | 0.50 | 0.84 | 0.78 |
| 2 applications ML1 | 0.22 | 0.15 | 0.28 | 0.77 | 0.66 |
| 3 applications ML1 | 0.15 | 0.10 | 0.20 | 0.72 | 0.60 |
| 4 sprayed applications ML1 spray | 0.15 | 0.06 | 0.23 | 0.75 | 0.66 |
| 1 application ML2 (Comparative) | 0.18 | 0.10 | 0.25 | 0.67 | 0.46 |
| 2 applications ML2 | 0.13 | 0.07 | 0.19 | 0.62 | 0.39 |
| 3 applications ML2 | 0.09 | 0.05 | 0.14 | 0.56 | 0.32 |

### Saturated Application (Drop):

The overall transmission is higher for the multilayer material according to the invention ML1, in particular in the blue wavelength range; 57% as opposed to 39% for ML1 relative to the comparative ML2.

The multilayer material ML1 according to the invention functions better in terms of capacity for protecting against UV and of overall visible transparency than the material ML2 outside the invention.

Multiple Applications, Comparison between 1 Application and 3 Applications:

The overall UV transmission decreases greatly, notably for UVA; the transmission passes from 50% to 20% (reduction by a factor of 2.5) for the multilayer material ML1 according to the invention and from 25% to 13% (reduction by a factor of 1.9) for the comparative material ML2.

The overall visible transmission is significantly less impacted for the multilayer material ML1 of the invention than for the comparative multilayer material ML2, notably in the blue wavelength range: the transmission reduction is 1.3 for ML1 relative to a factor of 1.46 for ML2.

It follows that the multilayer material ML1 according to the invention has a better capacity for protecting against UV and better overall visible transparency than the multilayer material ML2 outside the invention.

### Sprayed Application:

It is seen that ML1 has good UV-screening properties and also high visible transmission.

### Analysis of the Transmittance-to-Wavelength Slope:

Transmittance-to-wavelength curves for the multilayer material according to the invention with λ the wavelength axis (nanometers) and t the transmittance axis (nm⁻¹):
t=0.0056λ−1.9155 (linear 1 drop ML1)
t=0.0034λ−0.7037 (linear 1 application ML1)
t=0.0048λ−1.4557 (linear 2 applications ML1)
t=0.0050λ−1.6315 (linear 3 applications ML1)
t=0.0055λ−1.765 (linear 4 applications as spray ML1)

Transmittance-to-wavelength curves for the multilayer material outside the invention:
t=0.0021λ−0.5468 (linear 1 drop ML2)
t=0.0019λ−0.4012 (linear 1 application ML2)
t=0.0017λ−0.3864 (linear 2 applications ML2)
t=0.0017λ−0.4491 (linear 3 applications ML2)

TABLE 10

| Slope of the curves | ML1 (Invention) | ML2 (outside the invention) |
|---|---|---|
| Application 1 saturated ″drop″ | 0.0056 | 0.0021 |
| 1 application | 0.0034 | 0.0019 |
| 2 applications | 0.0048 | 0.0017 |
| 3 applications | 0.0050 | 0.0017 |
| Sprayed 4 times | 0.0055 | / |

Values of ML1 and ML2 in table 10 are given in nm⁻¹

The UV and Visible transmittance-to-wavelength slope is obtained by linear regression; it is markedly higher for the multilayer material ML1 according to the invention than for the material ML2 outside the invention:

More than twice as high for ML1 in the saturated application and for an application versus ML2.

The slope parameter increases significantly with the number of applications for ML1, unlike ML2. The sprayed application also improves the slope parameter.

Multiple application of the comparative multilayer material ML2 affords little improvement as regards the slope parameter.

Besides the high transmittance in the visible range, of high transmittance-to-wavelength slope (greater than 3×10⁻³), the multilayer material of the invention has, as another noteworthy optical property, a narrow filtration front between UV and the visible range.

### Cut-Off Position

TABLE 11

| Cut-off position (nm) | ML1 (Invention) | ML2 (outside the invention) |
|---|---|---|
| Application 1 saturated ″drop″ | 405 | 481 |
| 1 application | 390 | 450 |
| 2 applications | 399 | 477 |
| 3 applications | 402 | 488 |
| Sprayed 4 times | 401 | / |

The cut-off position is well defined in the case of the multilayer material ML1 according to the invention at 400 nm±10 nm, independently of the application method. Conversely, in the case of the multilayer material ML2 outside the invention, the shift passes from 450 nm to 488 nm, which shows high dependence of the cut-off position as a function of the application method for the comparative ML2.

### Design and Simulation of Multilayer Materials

The following simulations will demonstrate designs fitting the invention description with other materials than the combination TiO₂/SiO₂.

### Description of the Silico Approach for the Design and Performance Evaluation

All designs composed of a material A and B presented in the following were achieved thanks to transfer matrix calculations coupled with a particle swarm optimization algorithm.

More precisely, the relationships between the refractive indices of materials A and B used and the thicknesses of the layers of each of these materials define the “cut-off position” of the transition profile of the transmission between the UVA wavelength range (320 nm to 400 nm) and the visible range (400 nm to 780 nm).

It is possible to model the thickness of the layers to optimize the optical properties.

The calculations linking the thicknesses and the refractive index of the (in)organic compounds A and B constituting the layers of the multilayer material of the invention with the optical properties (transmission, reflection, absorption) may notably be performed via the “Transfer Matrix Method” such as the one in the “open source” algorithms that are available, for example, at the address

https://fr.mathworks.com/matlabcentral/fileexchanpe/47637-transmittance-and-reflectance-spectra-of-multilavered-dielectric-stack-usinp-transfer-transfer-transfer-mansx-method.

According to a particular embodiment of the invention, the iterative calculations for optimizing the “cut-off” position are performed via a “particle swarm algorithm” from the optimization toolbox of the software Matlab from Mathworks company.

The refractive index data needed to model the optical properties of multilayers (real refractive index n and imaginary refractive index k) can be found in the open source database https://refractiveindex.info/. The specific references are reported in the following tabulation.

TABLE 12

| Material | Bibliographic references |
|---|---|
| BaF₂ | M. R. Querry. “Optical constants of minerals and other materials from the |
| | millimeter to the ultraviolet”, Contractor Report CRDEC-CR-88009 (1987) |
| CaCO₃ | G. Ghosh. “Dispersion-equation coefficients for the refractive index and |
| | birefringence of calcite and quartz crystals”, Opt. Commun. 163, 95-102 (1999) |
| | Additional comment: Since the material is berinfringent, the average of both |
| | extraordinary and ordinary refractive index is taken into account. Both data can |
| | be found in the previous reference. |
| MgF₂ | L. V. Rodríguez-de Marcos, J. I. Larruquert, J. A. Méndez, J. A. Aznárez. |
| | “Self-consistent optical constants of MgF2, LaF3, and CeF3 films”, Opt. |
| | Mater. Express 7, 989-1006 (2017) (Numerical data kindly provided by |
| | Juan Larruquert) |
| MgO | R. E. Stephens and I. H. Malitson. “Index of refraction of magnesium oxide”, |
| | J. Res. Natl. Bur. Stand. 49 249-252 (1952) |
| PS: | N. Sultanova, S. Kasarova and I. Nikolov. “Dispersion properties of optical |
| polystyrene | polymers”, Acta Physica Polonica A 116, 585-587 (2009) |
| TiO₂ | S. Sarkar, V. Gupta, M. Kumar, J. Schubert, P.T. Probst, J. Joseph, T.A.F. |
| | König, “Hybridized guided-mode resonances via colloidal plasmonic self- |
| | assembled grating”, ACS Appl. Mater. Interfaces, 11, 13752-13760 (2019) |
| | (Numerical data kindly provided by Dr. Tobias König) |
| SiO₂ | F. Lemarchand, private communications (2013). |
| ZnO | C. Stelling, C. R. Singh, M. Karg, T. A. F. König, M. Thelakkat, M. Retsch. |
| | “Plasmonic nanomeshes: their ambivalent role as transparent electrodes |
| | in organic solar cells”, Sci. Rep. 7, 42530 (2017)-see Supplementary |
| | information (Numerical data kindly provided by Tobias König) |
| ZnS | S. Ozaki and S. Adachi. “Optical constants of cubic ZnS”, Jpn. J. Appl. Phys. |
| | 32, 5008-5013 (1993) |

The surrounding medium simulates a cosmetic base of constant refractive index of value 1.45.

An ideal multi-application process was modelled to demonstrate the improvement of the optical performance as described in the invention. That is to say, we assume the stacking to be perfect so that a given multi-application of a given multilayer from the invention would be equivalent to another multilayer of higher number of layer from the invention. The equivalency tabulation is reported in the following table:

TABLE 13

| Type of multi-application | Multilayer equivalency |
|---|---|
| 3 layer multilayer applied once | 3 layers multilayer |
| 3 layers multilayer applied twice | 5 layers multilayer |
| 3 layers multilayer applied three times | 7 layers multilayer |
| 3 layers multilayer applied four times | 9 layers multilayer |
| 3 layers multilayer applied five times | 11 layers multilayer |
| 3 layers multilayer applied six times | 13 layers multilayer |
| 3 layers multilayer applied seven times | 15 layers multilayer |
| 3 layers multilayer applied eight times | 17 layers multilayer |
| 5 layers multilayer applied once | 5 layers multilayer |
| 5 layers multilayer applied twice | 9 layers multilayer |
| 5 layers multilayer applied three times | 13 layers multilayer |
| 5 layers multilayer applied four times | 17 layers multilayer |

Therefore, in order to demonstrate the optical performance improvement thanks to a simulated multi-application process, we will in the following directly compare the optical performances of 5, 9, 13, multilayers. The conclusions can be extrapolated from 3 to 17 layers.

### Validation of the Silico Performance Prediction

This section reproduces in simulation with the procedure described above the two experimental examples ML1 and ML2. Since the refractive index of the real and simulated materials are likely to be slightly different, the optimization of the ML S1 is slightly different from ML1

TABLE 14

| | | Simulated | | Experimental | |
|---|---|---|---|---|---|
| | | ML S1 | ML S2 | ML 1 | ML 2 |
| | | (invention) | (Outside invention) | (invention) | (Outside invention) |
| | Chemical | Layer Thickness | Layer Thickness | Layer Thickness | Layer Thickness |
| Layers | composition | (nm) | (nm) | (nm) | (nm) |
| 1 | TiO₂ | 18 | 32 | 21 | 32 |
| 2 | SiO₂ | 50 | 34 | 37 | 34 |
| 3 | TiO₂ | 36 | 67 | 42 | 67 |
| 4 | SiO₂ | 50 | 38 | 37 | 38 |
| 5 | TiO₂ | 18 | 22 | 21 | 22 |

### Results of Simulation

TABLE 15

| | | | | | | | Cut off |
|---|---|---|---|---|---|---|---|
| | | UV | UVB | UVA | Visible | Slope | position |
| | | 290-400 nm | 290-320 nm | 320-400 nm | 400-800 nm | (nm⁻¹) | (nm) |
| 1 application | Equivalent | 0.2895 | 0.0493 | 0.3757 | 0.9855 | 0.0082 | 380 |
| ML S1 | 5 layers | | | | | | |
| 2 applications | Equivalent | 0.0899 | 0.0075 | 0.1193 | 0.9736 | 0.0129 | 404 |
| ML S1 | 9 layers | | | | | | |
| 3 applications | Equivalent | 0.0338 | 0.0013 | 0.0454 | 0.9726 | 0.0226 | 405 |
| ML S1 | 13 layers | | | | | | |
| 1 application | Equivalent | 0.2948 | 0.0353 | 0.3901 | 0.9682 | 0.0058 | 380 |
| ML S2 | 5 layers | | | | | | |
| 2 applications | Equivalent | 0.1371 | 0.0036 | 0.1849 | 0.9274 | 0.0047 | 425 |
| ML S2 | 9 layers | | | | | | |
| 3 applications | Equivalent | 0.0897 | 0.0005 | 0.1213 | 0.9064 | 0.0041 | 435 |
| ML S2 | 13 layers | | | | | | |

Equation of Transition between UV and Visible Domain:
ML S1 x1 application: t(λ)=0.0082λ−2.5987 Spectral interval of validity: [325:450 nm]
ML S1 x2 applications: t(λ)=0.0129λ−4.6693 Spectral interval of validity: [355:445 nm]
ML S1 x3 applications: t(λ)=0.0226λ−8.6225 Spectral interval of validity : [375:425 nm]
ML S2 x1 application: t(λ)=0.0058λ−1.8847 Spectral interval of validity: [320:465 nm]
ML S2 x2 applications: t(λ)=0.0047λ−1.5688 Spectral interval of validity: [320:450 nm]
ML S2 x3 applications: t(λ)=0.0041λ−1.3972 Spectral interval of validity: [320:450 nm]

Both designs have similar performances for the simulation of an ideal application once, with
UV mean transmission respectively of 28.95% and 29.48% for ML S1 and MLS2,
UVA mean transmission respectively of 37.57% and 39.01% for ML S1 and MLS2,
UVB mean transmission respectively of 4.93% and 3.53% for ML S1 and MLS2,
Visible mean transmission respectively of 98.55% and 96.82% for ML S1 and MLS2,
   Multiple Applications, Comparison between 1 Application and 3 Applications:

The simulation of 3 applications in comparison to 1 application for each ML demonstrates:
Less impact in the visible range with a decrease of transmission of 1.3% for ML 51 against 6.3% for ML S2,
More efficiency In the UV with a decrease of transmission by a factor 8.6 for ML S1 against 3.3 for ML S2,
More efficiency In the UVA with a decrease of transmission by a factor 8.3 for ML S1 against 3.2 for ML S2,
More efficiency in the UVB with a decrease of transmission by a factor 70.6 for ML S2 against 38 for ML S21
The slope of the transition increases by a factor 2.8 for ML S1 and is quite constant for the design ML S2. It even slightly decreases by a factor 0.7.
The cut-off position stabilizes around 405 nm for ML S1 against 435 nm for ML S2.

Therefore, the first design (invention) is more efficient than the second (outside the invention). Regarding the diminution of the UV transmission, the constant behavior in the visible range, the respect of the cut-off position around 400 nm+/−10 nm and at last the augmentation of the transition slope between UV and visible domains.

### Experimental Data on Slope Parameter and Cut-Off Position Gathered on ML1 and ML2:

TABLE 16

| | Slope parameter (nm⁻¹) | Cut-off position |
|---|---|---|
| 1 application ML 1 | 0.0034 | 390 |
| 2 applications ML 1 | 0.0048 | 399 |
| 3 applications ML 1 | 0.0050 | 402 |
| 1 application ML 1 | 0.0019 | 450 |
| 2 applications ML 1 | 0.0017 | 477 |
| 3 applications ML 1 | 0.0017 | 488 |

### Between 1 Application and 3 Applications

The slope parameters increases by a factor 1.5 experimentally compared to a factor 2.8 in simulation respectively for ML1 and its simulated counterpart ML S1,
The slope parameter is quite constant both for ML2 and its simulated counterpart ML S2,
The cut-off position lies at 402 nm and 405 nm respectively for ML1 and its simulated counterpart ML S1.
The cut-off position of ML2 and its simulated counterpart ML S2 are both out of the invention specification respectively with values of 435 nm and 488 nm.

Although the values may be slightly different between simulated and experimental values due mainly to uncertainties on the true refractive index of the materials, the trends of performance are similar. Therefore we demonstrate that this performance prediction by simulation is in agreement with the experimental evaluation.

Exemplification with Other Materials than the Association TiO₂/SiO₂
Family A—with TiO₂

The thicknesses detailed and compositions of each layer are given in the following table:

TABLE 17

| | ML A1 | | ML A2 | | ML A3 | | ML A4 | |
|---|---|---|---|---|---|---|---|---|
| Thicknesses | In the | | In the | | In the | | In the | |
| (nm) | invention | | invention | | invention | | invention | |
| x | TiO₂ | 18 | TiO₂ | 17 | TiO₂ | 17 | TiO₂ | 15 |
| y | MgF₂ | 54 | BaF₂ | 62 | MgO | 55 | CaCO₃ | 60 |
| 2*x | TiO₂ | 36 | TiO₂ | 34 | TiO₂ | 34 | TiO₂ | 30 |

### Results of Simulation

TABLE 18

| | | | | | | | Cut off |
|---|---|---|---|---|---|---|---|
| | | UV | UVB | UVA | Visible | Slope | position |
| | | 290-400 nm | 290-320 nm | 320-400 nm | 400-800 nm | (nm⁻¹) | (nm) |
| 1 application | Equivalent | 0.2847 | 0.0401 | 0.3724 | 0.9849 | 0.0089 | 375 |
| ML A1 | 5 layers | | | | | | |
| 2 applications | Equivalent | 0.0930 | 0.0041 | 0.1246 | 0.9760 | 0.0131 | 400 |
| ML A1 | 9 layers | | | | | | |
| 3 applications | Equivalent | 0.0424 | 0.0005 | 0.0572 | 0.9764 | 0.0249 | 400 |
| ML A1 | 13 layers | | | | | | |
| 1 application | Equivalent | 0.2939 | 0.3763 | 0.0642 | 0.9830 | 0.0077 | 380 |
| ML A2 | 5 layers | | | | | | |
| 2 application | Equivalent | 0.0863 | 0.1126 | 0.0126 | 0.9703 | 0.0115 | 405 |
| ML A2 | 9 layers | | | | | | |
| 3 application | Equivalent | 0.0292 | 0.0386 | 0.0028 | 0.9690 | 0.0211 | 406 |
| ML A2 | 13 layers | | | | | | |
| 1 application | Equivalent | 0.4229 | 0.1071 | 0.5367 | 0.9902 | 0.0093 | 360 |
| ML A3 | 5 layers | | | | | | |
| 2 applications | Equivalent | 0. 2191 | 0.0387 | 0.2846 | 0.9809 | 0.0122 | 385 |
| ML A3 | 9 layers | | | | | | |
| 3 applications | Equivalent | 0.1281 | 0.0151 | 0.1696 | 0.9771 | 0.0145 | 395 |
| ML A3 | 13 layers | | | | | | |
| 1 application | Equivalent | 0.3376 | 0.0845 | 0.4285 | 0.9883 | 0.0077 | 370 |
| ML A4 | 5 layers | | | | | | |
| 2 applications | Equivalent | 0.1210 | 0.0243 | 0.1557 | 0.9756 | 0.0104 | 400 |
| ML A4 | 9 layers | | | | | | |
| 3 applications | Equivalent | 0.0482 | 0.0079 | 0.0629 | 0.9730 | 0.0169 | 405 |
| ML A4 | 13 layers | | | | | | |

Equation of Transition between UV and Visible Domain:
ML A1 x1 application: t(λ)=0.0089λ−2.8287 Spectral interval of validity: [325:440 nm]
ML A1 x2 applications: t(λ)=0.0131λ−4.7046 Spectral interval of validity: [350:440 nm]
ML A1 x3 applications: t(λ)=0.0249λ−9.4683 Spectral interval of validity: [375:420 nm]
ML A2 x1 application: t(λ)=0.0077λ−2.4018 Spectral interval of validity: [325:455 nm]
ML A2 x2 applications: t(λ)=0.0115λ−4.1129 Spectral interval of validity: [350:455 nm]
ML A2 x3 applications: t(λ)=0.0211λ−8.0554 Spectral interval of validity: [375:430 nm]
ML A3 x1 application: t(λ)=0.0093λ−2.8171 Spectral interval of validity: [300:415 nm]
ML A3 x2 applications: t(λ)=0.0122λ−4.1372 Spectral interval of validity: [330:415 nm]
ML A3 x3 applications: t(λ)=0.0145λ−5.0091 Spectral interval of validity: [345:415 nm]
ML A4 x1 application: t(λ)=0.0077λ−2.3296 Spectral interval of validity: [300:465 nm]
ML A4 x2 applications: t(λ)=0.0104λ−3.5811 Spectral interval of validity: [330:450 nm]
ML A4 x3 applications: t(λ)=0.0169λ−6.2607 Spectral interval of validity: [360:425 nm]
   Multiple Applications, Comparison between 1 Application and 3 Applications:

In each example ML A1, A2, A3, A4 between one application and 3 applications:
The transmission in the visible range has a variation within a 2% range,
The transmission in UV, decreases by a factor 6.7, 10.1, 3.3, 7 respectively for ML A1, A2, A3 and A4.
The transmission in UVB, decreases by a factor 80.2, 9.7, 7.1, 10.7 respectively for ML A1, A2, A3 and A4.
The transmission in UVA, decreases by a factor 6.5, 23, 3.2, 6.8 respectively for ML A1, A2, A3 and A4,
The slope parameter increases by a factor 2.8, 2.7, 1.56, 2.2 respectively for ML A1, A2, A3 and A4,
The cut-off position stabilizes for each design at 400 nm+/−6 nm.

In conclusion these four designs belonging to the definition of the invention demonstrates improvements of their optical properties (mean UV, UVA, UVB transmissions, slope of transition between UV and visible region and cut-off position) by a simulated multi-application process.

Family B—with Nb2O5

The thicknesses detailed and compositions of each layer are given in the following table:

TABLE 19

| | ML B1 | | ML B2 | | ML B3 | | ML B4 | |
|---|---|---|---|---|---|---|---|---|
| Thicknesses | In the | | In the | | In the | | In the | |
| (nm) | invention | | invention | | invention | | invention | |
| x | Nb₂O₅ | 13 | Nb₂O₅ | 16 | Nb₂O₅ | 14 | Nb₂O₅ | 14 |
| y | SiO₂ | 63 | MgF₂ | 57 | MgO | 57 | CaCO₃ | 63 |
| 2*x | Nb₂O₅ | 26 | Nb₂O₅ | 32 | Nb₂O₅ | 28 | Nb₂O₅ | 28 |

### Results of Simulation

TABLE 20

| | | | | | | | Cut off |
|---|---|---|---|---|---|---|---|
| | | UV | UVB | UVA | Visible | Slope | position |
| | | 290-400 nm | 290-320 nm | 320-400 nm | 400-800 nm | (nm⁻¹) | (nm) |
| 1 application | Equivalent | 0.2846 | 0.0663 | 0.3628 | 0.9817 | 0.0076 | 380 |
| ML B1 | 5 layers | | | | | | |
| 2 applications | Equivalent | 0.0804 | 0.0092 | 0.1055 | 0.9723 | 0.0128 | 404 |
| ML B1 | 9 layers | | | | | | |
| 3 applications | Equivalent | 0.0317 | 0.0017 | 0.0422 | 0.9732 | 0.0212 | 405 |
| ML B1 | 13 layers | | | | | | |
| 1 application | Equivalent | 0.2517 | 0.0467 | 0.3252 | 0.9787 | 0.0087 | 380 |
| ML B2 | 5 layers | | | | | | |
| 2 applications | Equivalent | 0.0648 | 0.0046 | 0.0861 | 0.9670 | 0.0135 | 404 |
| ML B2 | 9 layers | | | | | | |
| 3 applications | Equivalent | 0.0232 | 0.0005 | 0.0313 | 0.9662 | 0.0174 | 405 |
| ML B2 | 13 layers | | | | | | |
| 1 application | Equivalent | 0.4066 | 0.1246 | 0.5078 | 0.9892 | 0.0085 | 365 |
| ML B3 | 5 layers | | | | | | |
| 2 applications | Equivalent | 0.1885 | 0.0557 | 0.2345 | 0.9770 | 0.0114 | 395 |
| ML B3 | 9 layers | | | | | | |
| 3 applications | Equivalent | 0.1060 | 0.0281 | 0.1327 | 0.9733 | 0.0184 | 395 |
| ML B3 | 13 layers | | | | | | |
| 1 application | Equivalent | 0.3132 | 0.0990 | 0.3898 | 0.9837 | 0.0094 | 380 |
| ML B4 | 5 layers | | | | | | |
| 2 applications | Equivalent | 0.0954 | 0.0359 | 0.1155 | 0.9684 | 0.0163 | 404 |
| ML B4 | 9 layers | | | | | | |
| 3 applications | Equivalent | 0.0335 | 0.0150 | 0.0394 | 0.9654 | 0.0281 | 405 |
| ML B4 | 13 layers | | | | | | |

Equation of Transition between UV and Visible Domain:
ML B1 x1 application: t(λ)=0.0076λ−2.3661 Spectral interval of validity: [315:455 nm]
ML B1 x2 applications: t(λ)=0.0128λ−4.6086 Spectral interval of validity: [350:440 nm]
ML B1 x3 applications: t(λ)=0.0212λ−8.0269 Spectral interval of validity: [370:425 nm]
ML B2 x1 application: t(λ)=0.0087λ−2.8169 Spectral interval of validity: [325:455 nm]
ML B2 x2 applications: t(λ)=0.0135λ−4.9227 Spectral interval of validity: [350:455 nm]
ML B2 x3 applications: t(λ)=0.0174λ−6.5164 Spectral interval of validity: [375:430 nm]
ML B3 x1 application: t(λ)=0.0085λ−2.5457 Spectral interval of validity: [300:425 nm]
ML B3 x2 applications: t(λ)=0.0114λ−3.854 Spectral interval of validity: [325:425 nm]
ML B3 x3 applications: t(λ)=0.0184λ−6.318 Spectral interval of validity: [355:415 nm]
ML B4 x1 application: t(λ)=0.0094λ−3.002 Spectral interval of validity: [335:410 nm]
ML B4 x2 applications: t(λ)=0.0163λ−6.0363 Spectral interval of validity: [375:430 nm]
ML B4 x3 applications: t(λ)=0.0281λ−10.951 Spectral interval of validity: [325:455 nm]
   Multiple Applications, Comparison between 1 Application and 3 Applications:

In each example ML B1, B2, B3, B4 between one application and 3 applications:
The transmission in the visible range has a variation within a 2% range,
The transmission in UV, decreases by a factor 10.0, 10.8, 3.83, 9.34 respectively for ML B1, B2, B3 and B4.
The transmission in UVB, decreases by a factor 39, 93.4, 4.43, 6.6 respectively for ML B1, B2, B3 and B4.
The transmission in UVA, decreases by a factor 8.6, 10.4, 3.83, 9.89 respectively for ML B1, B2, B3 and B4,
The slope parameter increases by a factor 2.8, 2, 2.2, 3 respectively for ML B1, B2, B3 and B4,
The cut-off position stabilizes for each design at 400 nm +/−5 nm.

In conclusion these four designs belonging to the definition of the invention demonstrates improvements of their optical properties (mean UV, UVA, UVB transmissions, slope of transition between UV and visible region and cut-off position) by a simulated multi-application process.

Family C—with ZnO

The thicknesses detailed and compositions of each layer are given in the following table:

TABLE 21

| Thicknesses (nm) | ML C1 In the invention | |
|---|---|---|
| x | ZnO | 28 |
| y | MgF₂ | 62 |
| 2*x | ZnO | 56 |

### Results of Simulation

TABLE 22

| | | | | | | | Cut off |
|---|---|---|---|---|---|---|---|
| | | UV | UVB | UvA | Visible | Slope | position |
| | | 290-400 nm | 290-320 nm | 320-400 nm | 400-800 nm | (nm⁻¹) | (nm) |
| 1 application | Equivalent | 0.5653 | 0.4166 | 0.6205 | 0.9730 | 0.0043 | 375 |
| ML C1 | 5 layers | | | | | | |
| 2 applications | Equivalent | 0.3359 | 0.2168 | 0.3809 | 0.9474 | 0.0054 | 385 |
| ML C1 | 9 layers | | | | | | |
| 3 applications | Equivalent | 0.2010 | 0.1096 | 0.2363 | 0.9260 | 0.0076 | 400 |
| ML C1 | 13 layers | | | | | | |

Equation of Transition between UV and Visible Domain:
ML C1 x1 application: t(λ)=0.004λ−0.09309 Spectral interval of validity: [290:465 nm]
ML C1 x2 applications: t(λ)=0.0054λ−1.4895 Spectral interval of validity: [290:470 nm]
ML C1 x3 applications: t(λ)=0.00076λ−2.5319 Spectral interval of validity: [350:480 nm]
   Multiple Applications, Comparison between 1 Application and 3 Applications:

For example ML C1 between one application and 3 applications:
The transmission in the visible range stays above 96%,
The transmission in UV, decreases by a factor 1.7,
The transmission in UVB, decreases by a factor 3.8,
The transmission in UVA, decreases by a factor 2.6,
The slope parameter increases by a factor 1.77,
The Cut off stabilizes at 400 nm.

In conclusion this design belonging to the definition of the invention demonstrates improvements of its optical properties by a simulated multi-application process.

Family D—with ZnS

The thicknesses detailed and compositions of each layer are given in the following table:

TABLE 23

| Thicknesses (nm) | ML D1 In the invention | |
|---|---|---|
| x | ZnS | 7 |
| y | MgF₂ | 93 |
| 2*x | ZnS | 14 |

### Results of Simulation

TABLE 24

| | | | | | | | Cut off |
|---|---|---|---|---|---|---|---|
| | | UV | UVB | UVA | Visible | Slope | position |
| | | 290-400 nm | 290-320 nm | 320-400 nm | 400-800 nm | (nm⁻¹) | (nm) |
| 1 application | Equivalent | 0.4161 | 0.3031 | 0.4561 | 0.9332 | 0.0037 | 380 |
| ML D1 | 5 layers | | | | | | |
| 2 applications | Equivalent | 0.1573 | 0.1494 | 0.1563 | 0.8846 | 0.0074 | 405 |
| ML D1 | 9 layers | | | | | | |
| 3 applications | Equivalent | 0.0764 | 0.1042 | 0.0620 | 0.8442 | 0.0076 | 405 |
| ML D1 | 13 layers | | | | | | |

Equation of Transition between UV and Visible Domain:
ML D1 x1 application: t(λ)=0.0037λ−0.8667 Spectral interval of validity: [335:510 nm]
ML D1 x2 applications: t(λ)=0.0074λ−2.5171 Spectral interval of validity: [335:465 nm]
ML D1 x3 applications: t(λ)=0.0076λ−2.7247 Spectral interval of validity: [355:480 nm]
   Multiple Applications, Comparison between 1 Application and 3 Applications:

For example ML D1 between one application and 3 applications:
The transmission in the visible range stays above 84%,
The transmission in UV, decreases by a factor 5.4,
The transmission in UVB, decreases by a factor 2.9,
The transmission in UVA, decreases by a factor 7.35,
The slope parameter increases by a factor 2,
The Cut off stabilizes at 405 nm.

In conclusion this design belonging to the definition of the invention demonstrates improvements of its optical properties by a simulated multi-application process.

Family E—TiO₂ with Mix SiO₂/PS

In the particular case of a mix of organic and inorganic materials we simulated a mix of SiO₂ and polystyrene (PS) at a 10% wt concentration (mass fraction).

In order to simulate this mix we calculated the resulted n and k values of the new material:

n_{SIO290%PS10%}=0.9*n_{SiO2}+0.1*n_{PS}

k_{SIO290%PS10%}=0.9*k_{SiO2}+0.1*k_{PS}

The thicknesses detailed and compositions of each layer are given in the following table:

TABLE 25

| Thicknesses (nm) | ML E1 In the invention | |
|---|---|---|
| x | TiO₂ | 16 |
| y | SiO₂ (90%)/PS (10%) | 62 |
| 2*x | TiO2 | 32 |

### Results of Simulation

TABLE 26

| | | | | | | | Cut off |
|---|---|---|---|---|---|---|---|
| | | UV | UVB | UVA | Visible | Slope | position |
| | | 290-400 nm | 290-320 nm | 320-400 nm | 400-800 nm | (nm⁻¹) | (nm) |
| 1 application | Equivalent | 0.3234 | 0.0637 | 0.4164 | 0.9878 | 0.0088 | 370 |
| ML E1 | 5 layers | | | | | | |
| 2 applications | Equivalent | 0.1165 | 0.0099 | 0.1542 | 0.9815 | 0.0125 | 395 |
| ML E1 | 9 layers | | | | | | |
| 3 applications | Equivalent | 0.0588 | 0.0017 | 0.0790 | 0.9836 | 0.0210 | 400 |
| ML E1 | 13 layers | | | | | | |

Equation of Transition between UV and Visible Domain:
ML E1 x1 application: t(λ)=0.0088λ−2.7329 Spectral interval of validity: [305:415 nm]
ML E1 x2 applications: t(λ)=0.0125λ−4.415 Spectral interval of validity: [345:440 nm]
ML E1 x3 applications: t(λ)=0.0210λ−7.8267 Spectral interval of validity: [365:420 nm]
   Multiple Applications, Comparison between 1 Application and 3 Applications:

For example ML E1 between one application and 3 applications:
The transmission in the visible range stays above 98%,
The transmission in UV, decreases by a factor 5.5,
The transmission in UVB, decreases by a factor 37.5,
The transmission in UVA, decreases by a factor 5.3,
The slope parameter increases by a factor 2.4,
The Cut off stabilizes at 400 nm.

In conclusion this design belonging to the definition of the invention demonstrates improvements of its optical properties by a simulated multi-application process.

1. A multilayer material with an odd number N of layers:
comprising at least three layers, each layer of which consists of a material A or of a material B different from A, said successive layers A and B being alternated and two adjacent layers having different refractive indices;
for which the thickness of each layer obeys the mathematical formula (I) below: [x/y/(αx/y)/x]
in which formula (I):
x is the thickness of the inner and outer layer;
y is the thickness of the layer adjacent to the inner layer αx or the outer layer x;
α is an integer or fraction and α=2±0 to 15%,
the intermediate odd layers (αx) have a double thickness±0 to 15% of the thickness of said outer layers x; and
a represents an integer greater than or equal to 0, connected to the number of alternated layers N such that a=(N−3)/2;
it being understood that:
has a different thickness from y;
when several layers are of thickness x, this means that each layer has a thickness x±0 to 15%;
when several layers are of thickness y, this means that each layer has a thickness y±0 to 15%; and
when several layers are of thickness α x, this means that each layer has a thickness α x±0 to 15%.

2. The material as claimed in claim 1, which is free of substrate.

3. The material as claimed in claim 1, in which the adjacent layers x and y consist of (in)organic compounds with different refractive indices.

4. The material as claimed in claim 1, in which the materials A and B consist of inorganic materials that are pure or as a mixture; these inorganic compounds constituting A and B are chosen from:
germanium (Ge), gallium antimonide (GaSb), tellurium (Te), indium arsenide (InAs), silicon (Si), gallium arsenide (GaAs), indium phosphide (InP), gallium phosphide (GaP), graphite (C), chromium (Cr), zinc telluride (ZnTe), zinc sulfate (ZnSO₄), vanadium (V), arsenic selenide (As₂Se₃), rutile titanium dioxide (TiO₂), copper aluminum diselenide (CuAlSe₂), perovskite calcium titanate (CaTiO₃), tin sulfide (SnS), zinc selenide (ZnSe), anatase titanium dioxide (TiO₂), cerium oxide (CeO₂), gallium nitride (GaN), tungsten (W), manganese (Mn), titanium dioxide notably vacuum-deposited (TiO₂), diamond (C), niobium oxide (Nb₂O₃), niobium pentoxide (Nb₂O₅), zirconium oxide (ZrO₂), sol-gel titanium dioxide (TiO₂), zinc sulfide (ZnS), silicon nitride (SiN), zinc oxide (ZnO), aluminum (Al), hafnium oxide (HfO₂), corundum aluminum oxide or corundum (Al₂O), aluminum oxide (Al₂O₃), yttrium oxide (Y₂O₃), periclase magnesium oxide (MgO), polysulfone, sodium aluminum fluoride (Na₃AlF), lead fluoride (PbF₂), mica, aluminum arsenide (AlAs), sodium chloride (NaCl), sodium fluoride (NaF), silica (SiO₂), barium fluoride (BaF₂), potassium fluoride (KF), vacuum-deposited silica (SiO₂), indium tin oxide (ITO), strontium fluoride (SrF₂), calcium fluoride (CaF₂), lithium fluoride (LiF), magnesium fluoride (MgF₂), bismuth oxychloride (BiOCl), bismuth ferrite (BiFeO₃), and boron nitride (NB), and (bi)carbonate such as calcium carbonate (CaCO₃); compounds constituting A and B are more particularly chosen from particularly (TiO₂ or Nb₂O₅)+(SiO₂ or MgF₂ or BaF₂ or MgO or CaCO₃) and (ZnO or ZnS)+MgF₂.

5. The material as claimed in claim 1, in which materials A and/or B contain organic compounds chosen from polystyrene (PS), polycarbonate, urea formaldehyde, styrene-acrylonitrile copolymers, polyether sulfone (PES), polyvinyl chloride (PVC), polyamide nylons, styrene-butadiene copolymers, type II polyamide nylons, multiacrylic polymers, ionomers, polyethylene, polybutylene, polypropylene, cellulose nitrate, acetal homopolymers, methylpentene polymers, ethylcellulose, cellulose acetatebutyrate, cellulose propionate, cellulose acetate, chlorotrifluoroethylene (CTFE), polytetrafluoroethylene (PTFE), fluorocarbon or polyvinylidene fluoride (FEP).

6. The material as claimed in claim 1, in which the layers x consist of compounds with a higher refractive index than y being inorganic compounds.

7. The material as claimed in claim 1, in which the layers y consist of compounds with a lower refractive index than x chosen from metal oxides, halides and carbonates.

8. The material as claimed in claim 1, in which the layers y consist of compounds with a higher refractive index than x, and being inorganic compounds and are preferably chosen from metal oxides, particularly metal oxides of metals which are in the Periodic Table of the Elements in columns IIIA, IVA, VA, IIIB and lanthanides, more particularly chosen from the following metal oxides: TiO₂, CeO₂, Nb₂O₃, Nb₂O₅, HfO₂, Al₂O₃, Y₂O₃ and ZrO₂, more particularly TiO₂, Nb₂O₅, CeO₂ and preferentially TiO₂, Nb₂O₅ or TiO₂, CeO₂ and even more preferentially TiO₂.

9. The material as claimed in claim 1, in which the layers x consist of compounds with a lower refractive index than y chosen from metal oxides and halides.

10. The material as claimed in claim 1, in which the maximum thickness of each layer of the multilayer material is 120 nm.

11. The material as claimed in claim 1, in which a ranges from 0 to 7, (0≤a≤7; 3≤N≤17
it being understood that:
x has a different thickness from y;
when several layers are of thickness x, this means that each layer has a thickness x±0 to 15%;
when several layers are of thickness y, this means that each layer has a thickness y±0 to 15; and
when several layers are of thickness αx, this means that each layer has a thickness αx±0 to 15%.

12. (canceled)

13. (canceled)

14. A process for manufacturing the material as claimed claim 1, comprising the following steps:
1. preparing a substrate and optionally applying to the substrate at least one nonstick layer, also known as a sacrificial layer, onto said substrate;
2. depositing an odd number N of alternated layers of materials A and B consisting of (in)organic compounds of high and lower refractive index, or of low and higher refractive index, onto the substrate optionally coated with sacrificial layer;
3. detaching the multilayer material from the substrate optionally coated with sacrificial layer;
4. if necessary, adjusting the size of the multilayer material to obtain multilayer material particles; and
5. optionally performing a post-treatment optionally followed by a (re)adjustment.

15. The process as claimed in claim 14, in which the substrate consists of an inorganic compound.

16. The process as claimed in claim 11, which uses a nonstick or sacrificial layer, which is inert with respect to the substrate.

17. A composition comprising one or more multilayer materials as defined in claim 1.

18. A process for treating keratin materials by application to said materials of a composition as defined in claim 17, leaving to dry between the layers, the application(s) being sprayed or otherwise.

19. A process for protecting keratin materials against UVA and UVB which comprises applying to the keratin materials one or more multilayer materials as defined in claim 1.

20. The material as claimed in claim 1 in which the adjacent layers x and y consist of (in)organic compounds with different refractive indices differ by at least 0.3.

21. The material as claimed in claim 1, which includes between 3 and 17 layers and which is such that:

| | | Material | | | | | |
|---|---|---|---|---|---|---|---|
| | | Thickness of the layers x, y | | | | | |
| | | 3 | 5 | 7 | 9 | 13 | 17 |
| Layers | | layers | layers | layers | layers | layers | layers |
| 1 | A | x | x | x | x | x | x |
| 2 | B | y | y | y | y | y | y |
| 3 | A | x | αx | αx | αx | αx | αx |
| 4 | B | | y | y | y | y | y |
| 5 | A | | x | αx | αx | αx | αx |
| 6 | B | | | y | y | y | y |
| 7 | A | | | x | αx | αx | αx |
| 8 | B | | | | y | y | y |
| 9 | A | | | | x | αx | αx |
| 10 | B | | | | | y | y |
| 11 | A | | | | | αx | αx |
| 12 | B | | | | | y | y |
| 13 | A | | | | | x | αx |
| 14 | B | | | | | | y |
| 15 | A | | | | | | αx |
| 16 | B | | | | | | y |
| 17 | A | | | | | | x |

it being understood that:
x has a different thickness from y;
when several layers are of thickness x, this means that each layer has a thickness x±0 to 15%;
when several layers are of thickness y, this means that each layer has a thickness y±0 to 15%; and
when several layers have a thickness α x, this means that each layer a has a thickness α x±0 to 15%; and
x and y are the thicknesses of the layers of the material with x<y;
it being understood that the thicknesses of the layers x between each other, αx between each other and y between each other are identical, α being as defined previously.

22. The material as claimed in claim 1, which includes between 3 and 17 layers and which is such that:

| | | Material | | | | | |
|---|---|---|---|---|---|---|---|
| | | Thickness of the layers x, y | | | | | |
| | | 3 | 5 | 7 | 9 | 13 | 17 |
| Layers | | layers | layers | layers | layers | layers | layers |
| 1 | B | x | x | x | x | x | x |
| 2 | A | y | y | y | y | y | y |
| 3 | B | x | αx | αx | αx | αx | αx |
| 4 | A | | y | y | y | y | y |
| 5 | B | | x | αx | αx | αx | αx |
| 6 | A | | | y | y | y | y |
| 7 | B | | | x | αx | αx | αx |
| 8 | A | | | | y | y | y |
| 9 | B | | | | x | αx | αx |
| 10 | A | | | | | y | y |
| 11 | B | | | | | αx | αx |
| 12 | A | | | | | y | y |
| 13 | B | | | | | x | αx |
| 14 | A | | | | | | y |
| 15 | B | | | | | | αx |
| 16 | A | | | | | | y |
| 17 | B | | | | | | x |

Multilayer materials in which:
A and B are inorganic or organic materials of the adjacent layers with A having a higher refractive index than that of B; and
x and y are the thicknesses of the layers of the material such that x<y
it being understood that:
x is a different thickness from y; the thicknesses of layers x between each other, α x between each other and y between each other are identical, α being as defined previously;
when several layers are of thickness x, this means that each layer has a thickness x±0 to 15%;
when several layers are of thickness y, this means that each layer has a thickness y±0 to 15%; and
when several layers are of thickness α x, this means that each layer has a thickness α x±0 to 15%.
